(19) United States
(12) Patent Application Publication
(10) Pub. No.: US 2024/0016505 A1
(43) Pub. Date: Jan. 18, 2024

(54) SYSTEMS, APPARATUS AND METHODS FOR REMOVING AND FILTERING VESSEL OCCLUSIONS TO ISOLATE BLOOD FOR REINFUSION INTO A PATIENT

(71) Applicant: Retriever Medical, Inc., Irvine, CA (US)

(72) Inventors: Michael B Horowitz, Naples, FL (US); Benjamin William Bobo, Las Vegas, NV (US); Brandon Matthew Repko, Mars, PA (US)

(21) Appl. No.: 18/220,373

(22) Filed: Jul. 11, 2023

Related U.S. Application Data
(60) Provisional application No. 63/368,325, filed on Jul. 13, 2022.

(51) Int. Cl. A61B 17/22; A61M 5/165

(52) U.S. Cl. CPC A61B 17/22; A61M 5/165; A61B 2217/005

(57) ABSTRACT

A method for retrieving a mixture of clot material and blood from a vessel of a patient and isolating the blood from the mixture for reinfusion into the patient. The method including the step of providing a blood reinfusion system adapted to extract the mixture of clot material and blood from the vessel and isolating the blood for reinfusion into the subject.

### CROSS-REFERENCES TO RELATED APPLICATIONS

The present application claims the benefit of U.S. Pat. App. No. 63/368,325, filed on Jul. 13, 2022.

### FIELD OF THE INVENTION

The present invention relates generally to systems and methods for catheter-based removal of occlusions and unwanted matter from vessels, ducts and other cavities or lumens of an organism. More particularly, the present invention relates to systems, apparatus and methods for catheter-based removal and filtering of vessel occlusions, such as thrombi and clots, to isolate blood mixed therewith for reinfusion into a patient.

### BACKGROUND OF THE INVENTION

As is well established, a thrombus (or blood clot) is the product of blood coagulation typically due to poor circulation resulting from extended sedentary activity among other factors.

In the early stages of thrombus formation, a thrombus generally has a consistency that is soft and/or spongy. However, over time, a thrombus becomes more tough and fibrotic due to a biological phenomenon often referred to as a clotting cascade and biochemical crosslinking of fibrin molecules, wherein fibrotic tissues begin to grow into the thrombus, thereby forming a chronic thrombus that usually adheres to and scars a vessel wall.

A thrombus disposed in an arterial or venous vessel can, and often will, decrease blood flow through the affected vessel or, in some instances, completely terminate blood flow to a physiological structure, thereby inducing tissue ischemia and subsequent infarction of the physiological structure supplied with oxygenated blood by the vessel.

Infarction, however, does not develop or is greatly limited if the flow of blood to the physiological structure is reestablished rapidly. Failure to reestablish blood flow can, and often does, accordingly lead to irreversible hypoxic damage to the physiological structure, angina pectoris, myocardial infarction, stroke, or even death.

Further, a thrombus can also fragment and release free, circulating portions of the thrombus, i.e., emboli, which circulate through arterial or venous vessels and occlude further vessels downstream of the thrombus. Emboli can often cause an embolism, or an occlusion of vessels, which can similarly lead to a stroke, heart attack, pulmonary embolism, etc.

Various conventional treatment systems (also referred to herein as “thrombectomy systems”) are now commercially available to remove thrombi, i.e., occlusions, from arterial and vascular vessel systems. Most conventional thrombectomy systems do, however, have numerous drawbacks and disadvantages that reduce their effectiveness, reliability, and ease of use.

One seminal disadvantage associated with conventional thrombectomy systems is that such systems are not appropriate for use in larger vascular structures such as the aorta, vena cava, and seminal peripheral vascular structures, such as the external iliac vein.

A further disadvantage associated with conventional thrombectomy systems is that such systems are not configured to effectively remove fibrous and/or calcified thrombi, i.e., chronic thrombi, as many thrombectomy systems necessitate the incorporation of an expandable wire structure into a thrombus, which is an exceptionally difficult process to conduct with chronic thrombi.

A further disadvantage associated with conventional thrombectomy systems is that such systems are generally not configured to effectively remove a thrombus from a stenotic vessel, due to a tendency for the stenotic vessel to strip and debride the thrombus from the system as it is retracted through a stenosis region.

A further disadvantage associated with conventional thrombectomy systems is that such systems typically require an operator to choose a predetermined interbody length, i.e., distance between system bodies on a delivery wire of a catheter system, at time of device insertion, which is often mis-sized by the operator and necessitates the possession of multiple catheter systems. As is well established, there are also increased manufacturing complexities and costs associated with the production of thrombectomy systems that include multiple catheter systems.

Indeed, conventional thrombectomy systems are also typically manufactured and deployed in a clinical setting with fixed diameter catheter systems that must be interchanged during a thrombectomy procedure, thereby introducing unnecessary complexity to the procedure and additional tissue trauma to a subject's vessel(s).

A further disadvantage associated with conventional thrombectomy systems is that such systems often also employ catheter systems that employ one or more fixed sized inflatable balloons for distal and/or proximal bodies and, thus, require that an operator pre-select a balloon size prior to deploying the catheter system in a vessel or cavity. Incorrect sizing of the inflatable balloons can also increase the probability of complications during the thrombectomy procedure, such as the disruption of a thrombus and subsequent release of emboli into a vessel.

A further disadvantage associated with conventional thrombectomy systems is that such systems often provide minimal, in any, distal protection from emboli and fail to catch or prevent emboli released into a vessel. Indeed, it is common for such systems to be incapable of capturing smaller emboli and, thus, allow the emboli to pass through the vessel.

Conventional thrombectomy systems have thus been produced that employ aspiration systems that are configured to aspirate thrombi and emboli from a vessel during a thrombectomy procedure and minimize the release of emboli into a vessel, such as AngioDynamics' AngioVac® System. There is, however, a seminal drawback associated with the use of aspiration systems, which is excessive blood loss, as aspiration systems often aspirate a substantial quantity of blood from a vessel along with thrombi and/or emboli. Blood loss associated with the use of aspiration systems is particularly pronounced when such systems are employed to aspirate thrombi and emboli from larger vessels, such as the aorta, and can necessitate blood transfusions.

Although autologous blood reinfusion systems have been developed to address the above noted blood loss associated with conventional thrombectomy systems, such as the Mari Medical FlowSaver® blood return system, such systems often only employ a single filter element that can be obstructed by emboli and sometimes fail to adequately filter blood for reinfusion.

It would thus be desirable to provide improved thrombectomy systems, apparatus and methods that substantially reduce or eliminate the disadvantages associated with conventional thrombectomy systems, apparatus and methods.

It is therefore an object of the invention to provide improved thrombectomy systems, apparatus and methods that substantially reduce or eliminate the disadvantages associated with conventional thrombectomy systems, apparatus and methods.

It is another object of the invention to provide improved thrombectomy systems and apparatus, and methods of using same, that are adapted to mechanically dislodge and remove occlusions from a vessel.

It is another object of the invention to provide improved thrombectomy systems, apparatus and methods that are adapted to mechanically dislodge and remove fibrous and/or calcified thrombi from a vessel.

It is another object of the invention to provide improved thrombectomy systems, apparatus and methods that provide optimal distal protection from emboli during removal of a thrombus or other occlusion from a vessel.

It is another object of the invention to provide improved thrombectomy systems, apparatus and methods that substantially minimize blood loss associated with aspirating thrombectomy systems.

It is another object of the invention to provide systems, apparatus and methods for removing occlusions from a subject's vessel and isolating blood mixed with occlusion material for reinfusion into the subject.

It is another object of the invention to provide systems, apparatus and methods for isolating blood mixed with occlusion material for reinfusion into the subject.

### SUMMARY OF THE INVENTION

The present application is directed to systems, apparatus and methods for removing and filtering vessel occlusions, such as thrombi and clots, to isolate blood mixed therewith for reinfusion into a patient.

In some embodiments, there is thus provided a method for retrieving a mixture of blood and clot material from a vessel of a patient, filtering the mixture to isolate the blood and reinfusing the filtered blood into the patient, comprising the steps of:
providing a retrieval system adapted to dislodge clots and associated clot material from a vessel of a patient;
providing an aspiration catheter configured and adapted to be positioned in a vessel of the subject;
providing a blood reinfusion system adapted to isolate the first blood from the mixture of the dislodged clot material and the first blood,
the blood reinfusion system comprising a top housing portion, the top housing portion of the blood reinfusion system comprising first and second ends, the first end of the top housing portion comprising a first inlet port configured to connect to the first suction source,
the blood reinfusion system further comprising a first reservoir adapted to receive the mixture of the dislodged clot material and the first blood,
a first perforated filter, the top housing portion of the blood reinfusion system adapted to allow the first perorated filter to be twisted in a first direction to be locked in a first position to the top housing portion, and twisted in a second direction, opposite to the first direction, to be unlocked from the first position,
a first intermediate housing portion comprising a third end and a fourth end,
a second filter positioned between the second end of the top housing portion of the blood reinfusion system and the third end of the first intermediate housing portion of the blood reinfusion system, wherein the second end of the top housing portion and the third end of the first intermediate housing portion are detachably attached,
a second intermediate housing portion comprising a fifth end and a sixth end,
a third filter positioned between the fourth end of the top housing portion of the blood reinfusion system and the fifth end of the second intermediate housing portion of the blood reinfusion system, wherein the fourth end of the top housing portion and the fifth end of the second intermediate housing portion are detachably attached,
a bottom housing portion comprising a seventh end and an eighth end, wherein the eighth end of the bottom housing portion comprises a second outlet port configured to connect to the second suction source,
a fourth filter positioned between the sixth end of the second intermediate housing portion and the seventh end of the bottom housing portion, wherein the sixth end of the second intermediate housing portion and the seventh end of the bottom housing portion are detachably attached,
a second reservoir, and
first and second suction sources;
dislodging clot material from the vessel with the retrieval system;
coupling the first suction source to the aspiration catheter;
positioning the aspiration catheter at a treatment site proximate the dislodged clot material within the vessel;
aspirating a mixture of the dislodged clot material and first blood from the vessel with the first suction source, wherein the mixture of the dislodged clot material and the first blood is introduced into and through the aspiration catheter and into the first suction source;
decoupling the first suction source from the aspiration catheter;
coupling the first suction source to the first inlet port of the blood reinfusion system;
injecting the mixture of the dislodged clot material and the first blood in the first suction source into and through the first inlet port and into the first reservoir of the blood reinfusion system, wherein the mixture of the dislodged clot material and the first blood passes through the first perforated filter of the blood reinfusion system and a first filtered blood is obtained, the first filtered blood passes through the second filter of the blood reinfusion system and a second filtered blood is obtained, the second filtered blood passes through the third filter and a third filtered blood is obtained, the third filtered blood passes through the fourth filter and a fourth filtered blood is obtained, and the fourth filtered blood passes into the second reservoir of the blood reinfusion system;
coupling the second suction source to the second outlet port of the blood reinfusion system; and
aspirating the fourth filtered blood from the second reservoir with the second suction source, wherein the fourth filtered blood is introduced into the second suction source for reinfusion into the patient.

In some embodiments, the first suction source comprises a syringe.

In some embodiments, the second suction source comprises one of a syringe or a pump.

In some embodiments, the first perforated filter has a pore in the range of approximately 1.0 mm to 5.0 mm.

In some embodiments, the second filter is of size in a range of approximately 200.0 micron to 1.0 mm, the third filter has a pore size in a range of approximately 40.0 micron to 1.0 mm, and the fourth filter has a pore size in a range of approximately 10.0 micron to micron.

In some embodiments, the blood reinfusion system further comprises a flow redirector element positioned above and proximate each of at least one of the first perforated filter, the second filter, the third filter, and the fourth filter.

In some embodiments, the retrieval system comprises an elongated member comprising a first proximal end, a first distal end, a first shaft, a second shaft, a third shaft, and a fourth shaft, wherein the first shaft is concentrically positioned around the second shaft, the second shaft is concentrically positioned around the third shaft, and the third shaft is concentrically positioned around the fourth shaft,
a proximal element releasably engaged to a first portion of the elongated member, whereby, when the proximal element is released from the elongated member, the proximal element is allowed to slidably translate along the elongated member, the first portion of the elongated member being disposed proximate the first proximal end of the elongated member,
the proximal element being adapted to expand from a first pre-expansion configuration to a first expanded configuration, and from the first expanded position to a first contracted configuration,
a distal element engaged to a second portion of the elongated member, the second portion of the elongated member being disposed proximate the first distal end of the elongated member,
the distal element adapted to expand from a second pre-expansion configuration to a second expanded configuration, and from the second expanded position to a second contracted configuration, and
a handle in physical communication with the elongated member, the handle comprising a first physically manipulable interface and a second physically manipulable interface,
the first physically manipulable interface adapted to selectably mechanically induce expansion of the proximal element from the first pre-expansion configuration to the first expanded configuration or the contraction of the proximal element from the first expanded position to the first contracted configuration or mechanically induce expansion of the distal element from the second pre-expansion configuration to the second expanded configuration, and from the second expanded position to the second contracted configuration when the first physically manipulable interface is moved,
the second physically manipulable interface adapted to induce slidable translation of the proximal element along the elongated member when the first proximal end point and the first distal end point of the proximal element are released from the elongated member and the second physically manipulable interface is moved.

In some embodiments, the first physically manipulable interface is configured to axially move the first shaft while the second, third and fourth shafts remain stationary.

In some embodiments, the first physically manipulable interface is configured to axially move the fourth shaft while said first, second and third shafts remain stationary.

In some embodiments, the second physically manipulable interface is configured to jointly move the first and second shafts axially while the third and fourth shafts remain stationary.

In some embodiments, there is provided another method for filtering clot material from blood to isolate the blood for reinfusion into a patient, comprising the steps of:
providing an aspiration catheter configured and adapted to be positioned in a vessel of the subject;
providing a blood reinfusion system adapted to isolate first blood from a mixture of dislodged clot material and the first blood,
the blood reinfusion system comprising a top housing portion, the top housing portion of the blood reinfusion system comprising first and second ends, the first end of the top housing portion comprising a first inlet port configured to connect to the first suction source,
the blood reinfusion system further comprising a first reservoir adapted to receive the mixture of the dislodged clot material and the first blood,
a first perforated filter, the top housing portion of the blood reinfusion system adapted to allow the first perorated filter to be twisted in a first direction to be locked in a first position to the top housing portion, and twisted in a second direction, opposite to the first direction, to be unlocked from the first position,
a first intermediate housing portion comprising a third end and a fourth end,
a second filter positioned between the second end of the top housing portion of the blood reinfusion system and the third end of the first intermediate housing portion of the blood reinfusion system, wherein the second end of the top housing portion and the third end of the first intermediate housing portion are detachably attached,
a second intermediate housing portion comprising a fifth end and a sixth end,
a third filter positioned between the fourth end of the top housing portion of the blood reinfusion system and the fifth end of the second intermediate housing portion of the blood reinfusion system, wherein the fourth end of the top housing portion and the fifth end of the second intermediate housing portion are detachably attached,
a bottom housing portion comprising a seventh end and an eighth end, wherein the eighth end of the bottom housing portion comprises a second outlet port configured to connect to the second suction source,
a fourth filter positioned between the sixth end of the second intermediate housing portion and the seventh end of the bottom housing portion, wherein the sixth end of the second intermediate housing portion and the seventh end of the bottom housing portion are detachably attached,
a second reservoir, and
first and second suction sources;
coupling the first suction source to the aspiration catheter;
positioning the aspiration catheter at a treatment site proximate the clot material within the vessel;
aspirating a mixture of the clot material and the first blood from the vessel with the first suction source, wherein the mixture of the clot material and the first blood is introduced into and through the aspiration catheter and into the first suction source;
decoupling the first suction source from the aspiration catheter;
coupling the first suction source to the first inlet port of the blood reinfusion system;
injecting the mixture of the clot material and the first blood in the first suction source into and through the first inlet port and into the first reservoir of the blood reinfusion system, wherein the mixture of the clot material and the first blood passes through the first perforated filter of the blood reinfusion system and a first filtered blood is obtained, the first filtered blood passes through the second filter of the blood reinfusion system and a second filtered blood is obtained, the second filtered blood passes through the third filter and a third filtered blood is obtained, the third filtered blood passes through the fourth filter and a fourth filtered blood is obtained, and the fourth filtered blood passes into the second reservoir of the blood reinfusion system;
coupling the second suction source to the second outlet port of the blood reinfusion system; and
aspirating the fourth filtered blood from the second reservoir with the second suction source, wherein the fourth filtered blood is introduced into the second suction source for reinfusion into the patient.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 depicts a deployment stage of an embodiment of the retrieval apparatus, showing the proximal and distal bodies deployed on a delivery wire, in accordance with the invention;

FIG. 2 depicts the pre-deployment stage of the proximal and distal bodies shown in FIG. 1, where the proximal and distal bodies are restrained or unexpanded, in accordance with the invention;

FIG. 3 depicts the expanded or released configuration of the proximal and distal bodies of the retrieval apparatus shown in FIG. 2 after from the delivery catheter, in accordance with the invention;

FIG. 4 depicts advancement of the proximal body of the retrieval apparatus shown in FIG. 3 axially along the delivery wire to trap and compress a thrombus, in accordance with the invention;

FIG. 5 depicts the capture of a thrombus using the deployed proximal and distal bodies of the retrieval apparatus shown in FIG. 3, in accordance with the invention;

FIG. 6 depicts a thrombus lodged in an artery, in accordance with the invention;

FIG. 7 depicts a deployment stage of another embodiment of a retrieval apparatus, in accordance with the invention;

FIG. 8 depicts the guide catheter of the retrieval apparatus shown in FIG. 7 with an inflated balloon adapted to arrest anterograde blood flow through the vessel, in accordance with the invention;

FIG. 9 depicts the delivery catheter shown in FIG. 8 positioned proximate to the thrombus and the delivery wire through the thrombus, in accordance with the invention;

FIG. 10 depicts the delivery catheter shown in FIG. 9 advanced over the delivery wire and through the thrombus, in accordance with the invention;

FIG. 11 further depicts the delivery catheter shown in FIGS. 9 and 10 advanced through the delivery catheter, with the proximal and distal bodies restrained in the delivery catheter, in accordance with the invention;

FIG. 12 depicts the delivery catheter shown in FIG. 11 withdrawn proximally and the proximal and distal mesh bodies deployed and positioned proximal and distal to the thrombus, in accordance with the invention;

FIG. 13 depicts the proximal body shown in FIG. 12 released from the delivery wire and advanced axially wherein the proximal and distal bodies trap and compress the thrombus between the proximal and distal bodies, in accordance with the invention;

FIG. 14 depicts the delivery catheter shown in FIG. 13 with the bodies and compressed thrombus being removed as a single unit while suction is applied to the guide catheters with an aspiration system, in accordance with the invention;

FIG. 15 depicts another embodiment of a retrieval apparatus comprising an active segment having a section spanning the length of a thrombus, and a delivery segment containing one embodiment of an incorporation structure, in accordance with the invention;

FIG. 16 depicts another embodiment of a retrieval apparatus comprising an active segment having a section spanning the length of a thrombus, and a delivery segment containing another embodiment of an incorporation structure, in accordance with the invention;

FIG. 17A depicts another embodiment of a retrieval apparatus comprising a single tether arrangement attached to a proximal body, in accordance with the invention;

FIG. 17B depicts another embodiment of a retrieval apparatus comprising a double tether arrangement attached to the proximal body, in accordance with the invention;

FIG. 18 depicts a deployment stage of an embodiment of a retrieval apparatus, showing the placement of a guide catheter with a catheter balloon into common carotid artery, in accordance with the invention;

FIG. 19 depicts the advancement of an access catheter of the retrieval apparatus shown in FIG. 18 into a vessel, in accordance with the invention;

FIG. 20 depicts the advancement of a delivery catheter and delivery wire catheter of the retrieval apparatus shown in FIG. 18 to the origin of an occlusion, in accordance with the invention;

FIG. 21 depicts inflation of the catheter balloon of the retrieval apparatus shown in FIG. 18 to arrest anterograde flow, in accordance with the invention;

FIG. 22 depicts the advancement of the delivery catheter shown in FIG. 20 through the thrombus with the proximal and distal bodies compressed therein, in accordance with the invention;

FIG. 23 depicts deployment of the proximal and distal bodies of the delivery catheter shown in FIGS. 20 and 22 and disposed distal to the thrombus, in accordance with the invention;

FIG. 24 depicts the proximal and distal bodies shown in FIG. 22 drawn proximally to the thrombus and expanding to a larger diameter proximate to the M1 and M2 branches, in accordance with the invention;

FIG. 25 depicts the proximal body shown in FIGS. 23 and 24 moved proximally by the tether, in accordance with the invention;

FIG. 26 depicts the thrombus withdrawn towards the access catheter, in accordance with the invention;

FIG. 27 depicts removal of the thrombus through an aspiration catheter with suction, in accordance with the invention;

FIG. 28A depicts a perspective view of another embodiment of a retrieval apparatus, in accordance with the invention;

FIG. 28B depicts a partial side plan view of a distal tip portion of the retrieval apparatus shown in FIG. 28A, showing the proximal and distal mesh structure elements or bodies, in accordance with the invention;

FIG. 28C depicts a side plan view of another embodiment of a proximal mesh structure element of the retrieval apparatus shown in FIG. 28A, in accordance with the invention;

FIG. 28D depicts a front plan view of the proximal element shown in FIG. 28C, in an expanded configuration, in accordance with the invention;

FIG. 28E depicts a partial side plan view of a distal element assembly of the device shown in FIG. 28A, in accordance with the invention;

FIG. 28F depicts a front plan view of the distal element shown in FIG. 28E in an expanded configuration, in accordance with the invention;

FIG. 28G depicts partial side plan view of the retrieval apparatus shown in FIG. 28A, in accordance with the invention;

FIG. 28H depicts a perspective view of another embodiment of a retrieval apparatus, in accordance with the invention;

FIG. 28I depicts a perspective view of one embodiment of an aspiration syringe, in accordance with the invention;

FIG. 29A depicts a cross-sectional view of one embodiment of a blood reinfusion system, in accordance with the invention;

FIG. 29B is a side plan cross-sectional view of a top housing portion of the blood reinfusion system shown in FIG. 29A, in accordance with the invention;

FIG. 29C is a top plan cross-sectional view of a top housing portion of the blood reinfusion system shown in FIG. 29A, in accordance with the invention;

FIG. 29D is a side plan cross-sectional view of a flow director element of the blood reinfusion system shown in FIG. 29A, in accordance with the invention;

FIG. 29E is another side plan cross-sectional view of a flow director element of the blood reinfusion system shown in FIG. 29A, in accordance with the invention; and

FIG. 30 is a perspective view of another embodiment of a blood reinfusion system, in accordance with the invention.

### DETAILED DESCRIPTION OF THE PREFERRED EMBODIMENT

Before describing the present invention in detail, it is to be understood that this invention is not limited to particularly exemplified systems, apparatus, structures or methods as such may, of course, vary. Thus, although a number of systems, apparatus, structures and methods similar or equivalent to those described herein can be used in the practice of the present invention, the preferred systems, apparatus, structures and methods are described herein.

Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed.

Unless defined otherwise, all technical and scientific terms used herein have the same meaning as commonly understood by one having ordinary skill in the art to which the invention pertains.

For purpose of clarity, details relating to technical material that is known in the technical fields related to the invention have not been described in detail so as not to unnecessarily obscure the present invention.

It is understood that language used in this specification should not be interpreted as a general disavowal of any one specific embodiment or used to limit the claims beyond the meaning of the terms used therein.

It is also understood that the general principles defined herein can be applied to other embodiments and applications without departing from the spirit and scope of the invention.

It is also understood that any feature or component described in association with a specific embodiment may be used and implemented with any other embodiment unless clearly indicated otherwise.

As used in this specification and the appended claims, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items.

As also used in this specification and the appended claims, the singular forms “a”, “an” and “the” include plural referents unless the content clearly dictates otherwise.

Further, ranges, as used in this specification, can be expressed herein as from “about” or “approximately” one particular value, and/or to “about” or “approximately” another particular value. When such a range is expressed, another embodiment includes from the one particular value and/or to the other particular value. Similarly, when values are expressed as approximations, by use of the antecedent “about” or “approximately”, it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “about” or “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed.

Further, all publications, patents and patent applications cited herein, whether supra or infra, are hereby incorporated by reference in their entirety.

### Definitions

The terms “element”, “body” and “member” are used interchangeably herein in connection with the retrieval systems and apparatus of the invention, and mean and include permeable, partially impermeable and impermeable structures adapted to communicate with delivery wires and elongated delivery members of the retrieval systems and apparatus.

The terms “clot”, “thrombus”, “occlusion”, “vessel occlusion”, “blockage” and “foreign body” are also used interchangeably herein and mean and include unwanted or undesired material in a patient's veins or arteries that is partially or completely obstructing the flow of blood, or unwanted or undesired material disposed in any vessel in a subject's body that is partially or completely obstructing the flow of any fluid, such as urine, therethrough.

The terms “clot”, “thrombus”, “occlusion”, “vessel occlusion”, “blockage” and “foreign body” thus mean and include, without limitation, acute thrombi, subacute thrombi and chronic thrombi of arterial and venous structures.

The terms “one embodiment”, “one aspect”, and “an embodiment” and “an aspect”, as used herein, mean that a particular feature, structure, or characteristic described in connection with the embodiment can be included in at least one embodiment and not that any particular embodiment is required to have a particular feature, structure or characteristic described herein unless set forth in the claim.

The phrase “in one embodiment” or similar phrases employed herein do not limit the inclusion of a particular element of the invention to a single embodiment. The element can thus be included in other, or all embodiments discussed herein.

The term “substantially”, as used herein, means and includes the complete or nearly complete extent or degree of an action, characteristic, property, state, structure, item, or result to function as indicated. For example, an object that is “substantially” enclosed would mean that the object is either completely enclosed or nearly completely enclosed. The exact allowable degree of deviation from absolute completeness may in some cases depend on the specific context, such that enclosing nearly all the length of a lumen would be substantially enclosed, even if the distal end of the structure enclosing the lumen had a slit or channel formed along a portion thereof.

Use of the term “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

The term “comprise” and variations of the term, such as “comprising” and “comprises,” means “including, but not limited to” and is not intended to exclude, for example, other components, elements or steps.

The following disclosure is provided to further explain in an enabling fashion the best modes of performing one or more embodiments of the present invention. The disclosure is further offered to enhance the understanding and appreciation for the inventive principles and advantages thereof, rather than to limit in any manner the invention. The invention is defined solely by the appended claims, including any amendments made during the pendency of this application, and all equivalents of those claims as issued.

As indicated above, the present invention relates to retrieval systems and apparatus adapted and configured to remove occlusions and unwanted matter, such as a thrombus or clot, from an artery, vein, duct, or other cavity or lumen of an organism.

The present invention also relates to systems, apparatus and methods for filtering of vessel occlusions, such as thrombi and clots, mixed with blood to isolate the blood for reinfusion into a patient.

According to the invention, the retrieval apparatus can be used as a foreign body retriever and as a thrombectomy device in the arterial, venous and neural systems.

In various embodiments, the retrieval systems and apparatus are configured to be used in all venous structures: including dural venous sinuses, coronary arteries, cardiac chambers, all arteries, all ducts, ureters, urethra and fistulas.

It should be appreciated that each of the retrieval system and apparatus embodiments disclosed herein can be used in one or more of the following medical procedures:
a) septal heart repairs in which a catheter is manipulated to the patient's heart in order to permanently place two connected patches in a hole to cover both the left and right atrial sides;
b) pulmonary embolisms in which a catheter is manipulated proximate a blood clot that is lodged in an artery in the lung thereby blocking blood flow to part of the lung;
c) deep vein thrombosis in which a catheter is manipulated proximate a blood clot that is lodged in a vein, often in the patient's legs, thereby blocking blood flow;
d) repairing fistulas;
e) removing fistula thrombi;
f) removing stones from the patient's gastrointestinal & hepaticobiliary system;
g) removing stones from the patient's genitourinary system;
h) removing foreign bodies from any location in the patient's body; and
i) filter thrombus removal.

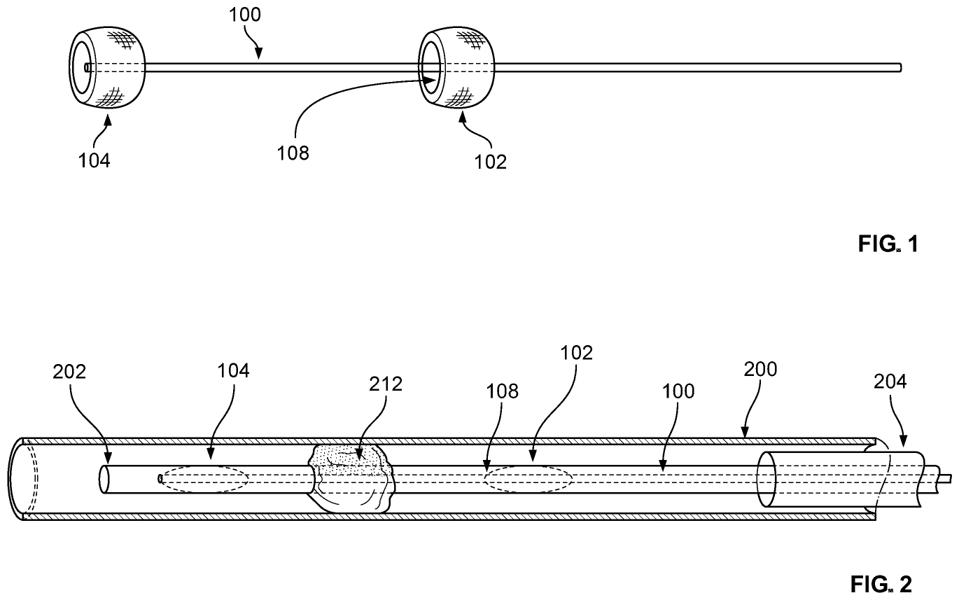

Referring now to FIG. 1, there is depicted one embodiment of a deployed retrieval apparatus comprising a distal body 104, which, in this embodiment, comprises a body permanently affixed and, hence, mounted to the delivery wire 100.

Referring to all embodiments disclosed herein, it should be noted that prior to deployment of the delivery wire 100, a guide wire can be used to position any element of the system disclosed herein, including a delivery catheter (also referred to interchangeably herein as a “microcatheter”) 202, guide catheter 204, and delivery wire 100 into the preferred position within a vessel or other interior.

The “bodies” (or “elements”) referred to herein can comprise a mesh, and they can comprise a nickel-titanium (Ni) alloy, such as Nitinol™ or other suitable expandable biocompatible material. The mesh construct of the distal and proximal bodies 104, 102 can, and often will, reduce the risk of distal embolization of portions of a clot since the mesh construct can capture embolic material within its interstices.

The distal body 104 can, in some embodiments, have differently-sized mesh and/or can comprise a selectively permeable material, or it may be non-permeable.

In some embodiments, the proximal body 102 is temporarily affixed to and, hence, mounted to the delivery wire 100. The temporary affixed aspect referred to above is releasable such that upon release the proximal body 102 can move along the wire, which is referred to herein as “axial movement” along the delivery wire 100, while remaining in communication with the wire 100. This aspect is referred to herein as being in “releasable engagement” or being “releasably engaged” to the delivery wire 100.

Such releasable engagement can be achieved, for example, by using breakable connection 108, which in some embodiments, can be an electrolytically or heat removable/disconnectable connection or mechanical connection that can be selectively disconnected by the clinician.

By way of example, in the case of an electrolytically or heat removable/disconnectable connection, the clinician can apply a current to the connection, (in embodiments via the delivery wire 100 which can be conductive) wherein the electrical current breaks or melts the connection.

According to the invention, the connection can include, without limitation, a breakable connection 108, linking a proximal body 102 to the delivery wire 100, which can be eroded and/or disintegrated through the application of electrical current.

According to the invention, the breakable connection 108 can be preloaded onto the retrieval device in order to secure the proximal body 102 in a preferred location and/or configuration.

According to the invention, the breakable connection can have a plurality of shapes and designs, including, but not limited to, a straight post extending from the delivery wire 100 to the proximal 102 or other body, a loop configuration of the breakable connection passing through the material of the proximal 102 or other body, and/or a “nail” configuration in which a straight post extends from the delivery wire to the proximal 102 or other body, wherein the post has an enlarged end, or nail head, within the body that can be eroded by the application of electric current to release the body.

In some embodiments of the invention, the proximal body 102 or other element can be secured to the delivery wire 100 using more than one breakable connection 108.

By way of example, a proximal body 102 can be secured with multiple breakable connections, each having a different length and a different release threshold, allowing the breakable connections to be sequentially released from the delivery wire 100.

In some embodiments, more than one proximal body 102 is secured to the delivery wire 100 using a breakable connection 108.

According to the invention, melting of a breakable connection can be caused by the application of electrical current, fluid, and/or chemical compounds. Melting can occur in a physical member that is used to secure the proximal or distal body and/or may occur within an adhesive that binds the physical member to the proximal body 102, and/or the delivery wire 100.

According to the invention, breakable connection techniques and methods, including, but not limited to, those shown in U.S. Pat. Nos. 5,683,451, 5,855,578, 6,245,076, 8,273,116 and U.S. Pub. Nos. 2007/0100414, 2009/0062726, and 2010/0268251, can be used to release a proximal body and/or distal body, as described herein. In the case of a mechanically breakable connection, the breakable connection 108 can be made of a suture, brace, thread or other material that is able to be broken upon application of force to the breakable connection 108.

In some embodiments, the distal motion of a catheter, such as the delivery catheter 202 shown in FIG. 2, with a force above the threshold holding force of the breakable connection 108 causes the connection 108 to break or release, thus allowing the proximal body 102 to move along the delivery wire 100 in the manners described herein. The “bodies” (or “elements”) referred to herein may be of various geometric shapes including a disc or sphere.

According to the invention, the distal body 104 and/or proximal body 102 can comprise an inflatable device, including, but not limited to, an inflatable balloon. In some embodiments, a retrieval device, as described herein, includes a distal body 104 and a proximal body 102 made of differing materials. By way of example, a proximal body 102 can be an inflatable balloon and a distal body 104, on the same retrieval device, can be made of a mesh material.

According to the invention, by adjusting the manufactured radial force, body diameter, and strength of the bodies, foreign body extraction, as described herein, can also be used for the removal of stones, pulmonary emboli, or some other type of obstruction.

According to the invention, in some embodiments, a proximal body 102 and/or distal body 104 can have variable radial force, or stiffness across sub-regions of the body itself. By way of example, the upper hemisphere of a spherical body can have a difference radial force characteristic than the lower hemisphere of the body.

In some embodiments, the proximal and distal bodies 102, 104 are substantially the same.

In some embodiments, the proximal and distal bodies 102, 104 are heterogeneous, having different compositions and characteristics including, without limitation, shape, size (e.g., thickness, diameter), configuration, pore size (e.g., mesh pore size), coating, or some other differing characteristic.

According to the invention, the proximal and/or distal bodies 102, 104 can comprise anti-platelet, or some other type of, coatings to reduce adhesion and provide a less thrombogenic environment during clinical application. The proximal and/or distal bodies 102, 104, and any material (e.g., wires) between these bodies, can also be coated with control release agents including, but not limited to, thrombolytic agents.

According to the invention, the delivery wire 100 referred to herein can comprise a wire structure or a hypo tube.

According to the invention, the delivery catheter 202 can form a plurality of shape configurations based on the clinical application in which it is used, for example, which type of vessel the delivery catheter is used within, the vessel size, the vessel shape, or some other application characteristic.

As depicted in FIG. 2, the delivery catheter 202 surrounds the delivery wire 100 and proximal and distal bodies 102, 104. As also depicted in FIG. 2, the proximal and distal bodies 102, 104 are restrained in the delivery catheter 202 in unexpanded configurations.

According to the invention, the delivery catheter 202 is adapted to pass through a vessel occlusion, such as a thrombus or clot 212. The proximal and distal bodies 102, 104 are also adapted to be released from the delivery catheter 202 either by retracting the delivery catheter 202 or advancing the wire 100, wherein, as discussed in detail below, the proximal and distal bodies 102, 104 transition to expanded configurations, as depicted in FIG. 1.

In a preferred embodiment, the distal body 104 remains fixed to the delivery wire 100, but the proximal body 102 (once released from its releasable engagement) can freely move along its axis and longitudinally along the delivery wire 100 when pushed by the delivery catheter 202, as depicted in FIG. 4.

The terms “pushed” and “pushing” as used herein in connection with moving a proximal body 102 or element along a delivery wire 100 means relative pushing. That is, the retraction of the delivery wire 100 while the delivery catheter 202 is kept in place can serve to move the proximal body 102 axially along the delivery wire 100. The terms “pushed” and “pushing” as is used herein thus refers to both forms of movement mentioned above.

According to the invention, once the proximal and distal bodies 102, 104 are positioned adjacent to both sides of the clot 212 (which has been referred to herein as “surrounded” or “surrounding” the clot) by movement of the proximal body 102, the clot 212 can be removed by retrieving the retrieval device from the vessel 200 and pulling the clot 212 free.

In some embodiments, movement of the proximal body 102 on the delivery wire 100 axially allows for compression of the clot 212 and obviates the need for pre-measuring or estimating the required distance between the distal and proximal bodies 102, 104 prior to entering the vessel 200, i.e., sizing may take place in situ within the vessel 200 upon the interventionist encountering it.

In some embodiments, mounting of the proximal body 102 on the delivery wire 100 occurs by running the delivery wire 100 through one of the mesh openings. In other embodiments, the proximal body 102 comprises an opening through which the delivery wire 100 can pass. In either case, the proximal body 102 is able to slide along the delivery wire 100 in an axial direction along the wire. This may be referred to herein as “slidably mounted.”

According to the invention, in some embodiments, the distal body 104 can also be slidably mounted in the way described above.

As described above, the proximal body 102 can be detachable (thus releasably engaged) using mechanical, electrolytic or some other type of control release format.

In some embodiments, the retrieval device comprises a distal body 104 and a proximal body 102, each of which, according to the invention, can be collapsible geometric forms.

Although the distal and proximal bodies 102, 104 depicted in FIG. 1 are presented for diagrammatic purposes as spherical, the distal and proximal bodies 102, 104 can also be other geometric forms such as disc, cone, oblong-shaped forms, etc.

As mentioned above, the distal and proximal bodies 102, 104 can be in the form of a mesh structure. The mesh structure provides several advantages over other material types, including, but not limited to, inflatable balloons. One significant advantage is that mesh structures are not susceptible to rupture via over inflation, such as an inflatable balloon.

As is well established, the clinical setting can also exacerbate complications related to the use of inflatable balloons within a lumen. By way of example, a calcified thrombus may increase the risk of balloon rupture.

According to the invention, the mesh cell size of the mesh structure can be manufactured to have different sizes based on factors such as the expected properties of the target foreign matter to be removed, such as the density of the matter.

In a preferred embodiment, the mesh structure of the proximal and distal bodies 102, 104 allows the bodies 102, 104 to expand upon release from the delivery wire 100 to the diameter and configuration of the cavity in which it is placed, such as a vessel 200 in which a thrombus 212 is located.

According to the invention, such mesh structures can comprise a shape memory nickel-titanium (Ni—Ti) alloy, such as Nitinol™, whereby, by way of example, the proximal and distal bodies 102, 104 can expand to a first dimension outside of a vessel 200 or catheter upon being subjected to a transition temperature, such as described in U.S. application Ser. No. 17/242,733, which is incorporated by reference herein.

The proximal and distal bodies 102, 104 can also be designed to expand to a continuum of smaller dimensions than the first dimensions corresponding to different lumen sizes. In this way, the proximal and distal bodies 102, 104 can fit the unique variations in diameter found in a lumen at the point of release and/or point of placement near an occlusion, such as a thrombus.

The mesh structure of proximal body 102 and/or distal body 104 also allows for improved distal flow during an intervention. The irregularity and/or texture of the expanded mesh structure may also facilitate the mesh structure becoming entangled or otherwise incorporated with a clot or occlusive substance, thereby increasing adhesion of the proximal body 102 and/or distal body 104 with the occlusion and facilitating its removal.

According to the invention, when the proximal body 102 is released, it can also be free to rotate on the delivery wire 100.

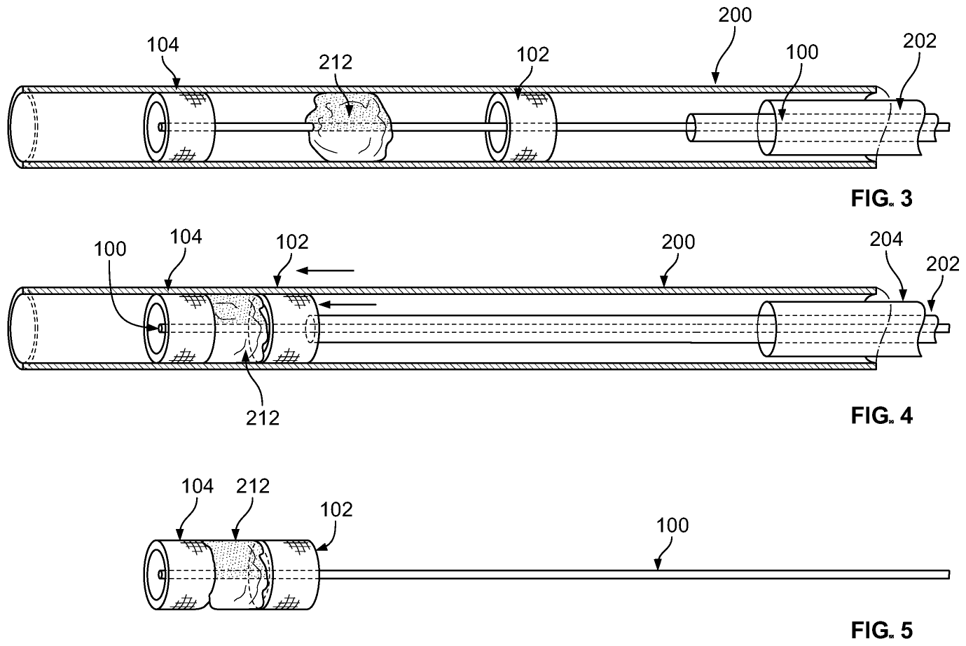

Referring now to FIG. 3, when the distal body 104 is placed distal to the target thrombus 212 for retrieval and the proximal body 102 is placed proximal to the thrombus 212, the distal and proximal bodies 104, 102 will straddle and contain the thrombus 212 intended for removal from the vessel 200. The proximal body 102 can now be advanced in the direction of the thrombus 212 in a variety of mechanical fashions.

As depicted in FIG. 4, a coaxially placed microcatheter, also referred to herein as a “delivery catheter” 202, can be pushed forward (once the proximal body is released) and be used to physically advance the proximal body 102 to ultimately capture and compress the thrombus 212.

Alternatively, the delivery catheter 202 can be used to hold the proximal body 102 in a fixed position while the delivery wire 100 is withdrawn thus moving the fixed distal body 104 towards the proximal body 102 and ultimately capturing and compressing the thrombus 212.

As shown in FIG. 5, once the thrombus 212 is captured/compressed between the distal body 104 and the proximal body 102, the entire retrieval device can be removed from the patient via withdrawal of the delivery wire 100 by, for example, withdrawing the proximal and distal bodies 102, 104 with the compressed thrombus 212 back to, and against, the delivery catheter 202 and then removing the delivery catheter 202, proximal and distal bodies 102, 104 and compressed material, i.e., thrombus 212, through the guide catheter 204. The guide catheter 204 can thereafter be withdrawn from the vessel 200.

As indicated above, the retrieval systems and apparatus of the invention can be employed to remove occlusions, such as a thrombus, from various arterial structures, i.e., preform an arterial thrombectomy. According to the invention, such a procedure can include the following generalized steps.

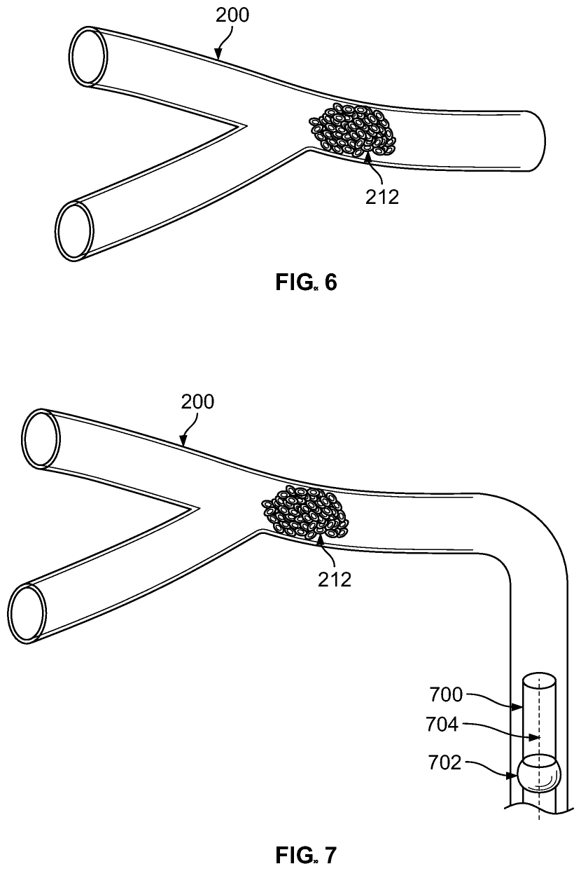

Referring first to FIG. 6, there is depicted an occlusion, such as a thrombus, 212 disposed in a vessel 200, in this instance, a proximal left internal carotid artery (ICA) of a subject.

FIG. 7 depicts an embodiment of a retrieval apparatus of the invention comprising a guide catheter 700 with a balloon 702 (“balloon catheter”). In some embodiments, suction may be applied through the guide catheter 700, in effect utilizing the guide catheter 700 as a suction catheter, as a given intervention may require. The balloon 702 is deflated and inserted into the ICA over a guide wire 704.

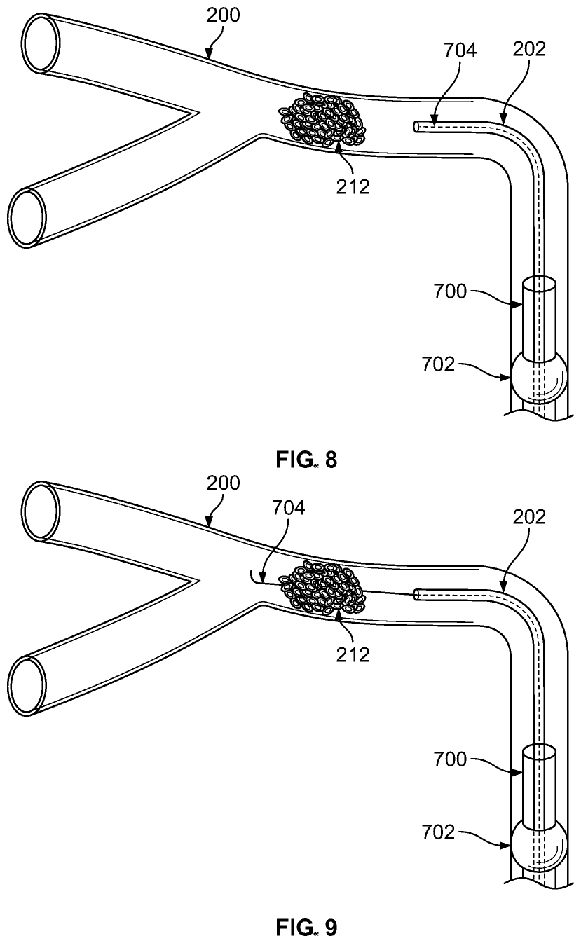

FIG. 8 depicts a delivery catheter 202 of the retrieval apparatus advanced through the balloon catheter 702 over the guide wire 704, which has been advanced.

FIG. 9 depicts a guide wire 704 of the retrieval apparatus being advanced through and distal to the occlusion 212.

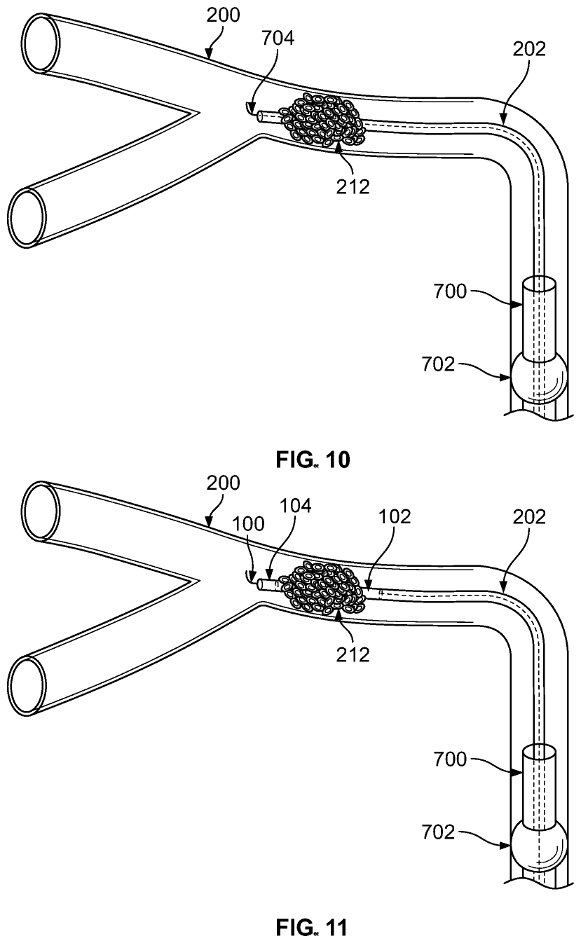

FIG. 10 depicts the delivery catheter 202 being advanced through and distal to the occlusion 212 (i.e., thrombus or clot) over the guide wire 704. The guide wire 704 is then removed (not shown).

FIG. 11 depicts the retrieval apparatus (with restrained proximal and distal bodies 102, 104 mounted on delivery wire 100) inserted into the delivery catheter 202.

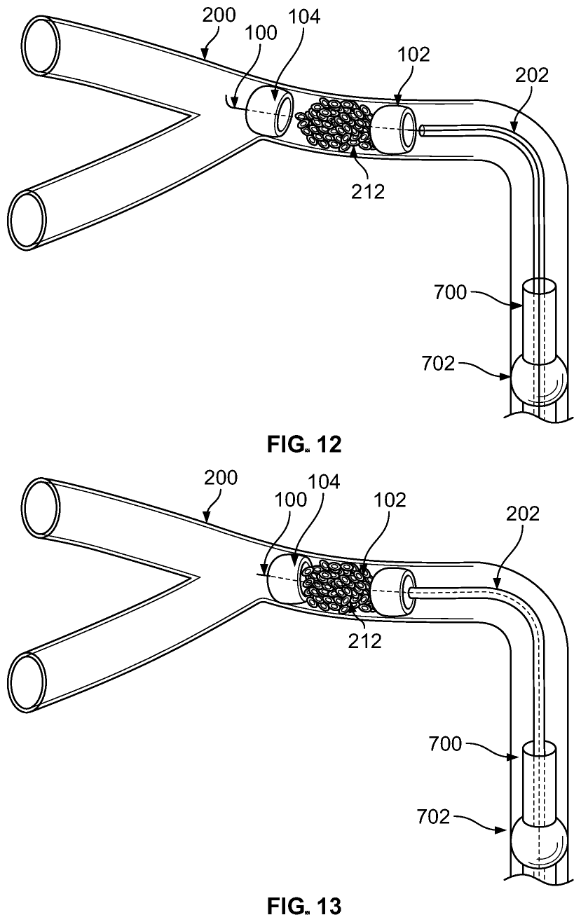

FIG. 12 depicts the delivery catheter 202 withdrawn (moved relative to the delivery wire 100) such that the proximal and distal bodies 102, 104 expand on either side of the occlusion 212.

FIG. 13 depicts the proximal body 102 released and advanced distally by force of the delivery catheter 202 (either by pushing the delivery catheter 202 or by pulling the delivery wire 100 while restraining the position of delivery catheter 202).

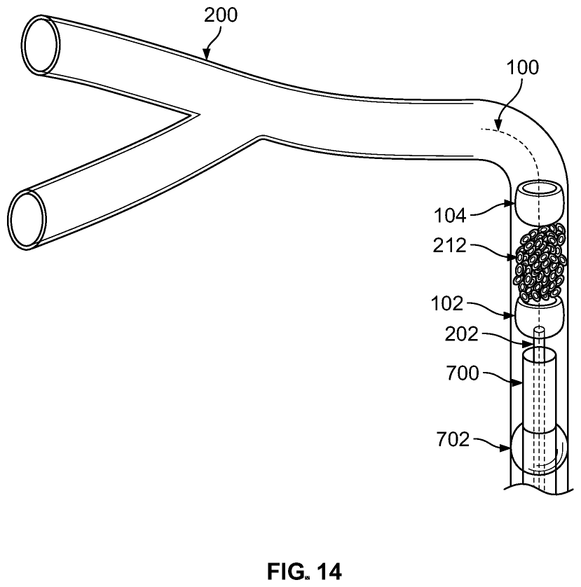

FIG. 14 depicts withdrawal of the delivery catheter 202 from the ICA, balloon 702 still inflated to arrest anterograde flow.

According to the invention, the retrieval apparatus can remove both organized and unorganized thrombi since, in some embodiments, the proximal and distal bodies 102, 104 of the retrieval apparatus do not need to be incorporated into the thrombus 212 to affect its removal.

According to the invention, the retrieval apparatus can also remove calcified, atherosclerotic material.

The retrieval apparatus can also be used centrally and peripherally by selecting the appropriate diameter and characteristics of the proximal and distal bodies 102, 104, such as appropriate radial force or stiffness, appropriate shape, whether the bodies 102, 104 are substantially identical or homogenous, mesh opening size in the bodies 102, 104, and the like.

According to the invention, the retrieval apparatus, as described herein, can have a plurality of sizes loaded within a common catheter, and a clinician may self-load, for example, different and/or additional proximal bodies, as described herein, rather than having to fully replace a deployed catheter for a second catheter-based device and system. This feature may reduce manufacturing costs and improve intervention efficiency.

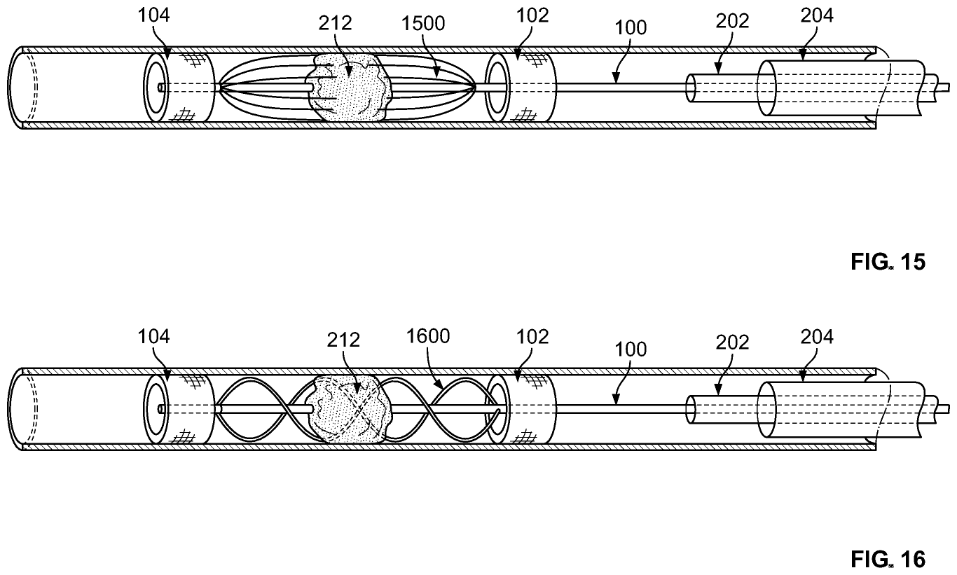

Referring now to FIGS. 15 and 16, there are depicted embodiments of the retrieval apparatus having a structure that is adapted and configured to incorporate into the thrombus 212, referred to herein as an “incorporation structure.”

In some embodiments, the incorporation structure is part of the delivery wire 100.

In some embodiments, the incorporation structure is separate.

As depicted in FIGS. 15 and 16, the delivery wire 100 (which in all embodiments disclosed herein may be a hypo tube) comprises a segment having characteristics different from that of the main segment of the delivery wire 100, or a structure mounted to the delivery wire 100 that may expand.

In the case where the incorporation structure is part of the delivery wire 100, the segment will be referred to herein as the “active segment” while the remainder of wire will be referred to as the “delivery segment.” The active segment is the segment having a section intended to span the length of the thrombus 212.

In some embodiments, the active segment comprises a cross-sectional shape that differs from the delivery segment.

In some embodiments, the delivery segment contains a suture material 1500 between the proximal and distal bodies 102, 104, such as depicted in FIG. 15.

According to the invention, the suture material 1500 gathers and moves along the delivery wire 100 as the proximal body 102 is advanced. Once the proximal body 102 is in position, the suture material 1500 is gathered in the area between the two bodies, which will enhance incorporation characteristics of the active area.

In some embodiments, the active area in the above noted example is the area between the two bodies 102, 104, which in this case, has suture material 1500 gathered therebetween.

As mentioned above, in some embodiments, the incorporation structure can comprise an additional expandable structure between the proximal and distal bodies 102, 104, which is adapted to expand and be incorporated into the thrombus 212.

According to the invention, the incorporation structure can comprise various mechanisms to enhance thrombus-incorporation, such as flanges, hooks, sutures, or some other material configuration. The incorporation structure can also comprise a sinusoidal wire 1600, such as depicted in FIG. 16.

According to the invention, prior to deployment of a retrieval apparatus, the distal body 104 can be affixed, mounted, adhered or otherwise connected to the delivery wire 100 or hypo tube in a collapsed or compressed state. Compression of the distal body 104 can be provided by the delivery catheter 202, and/or multiple catheters which surround the distal body 104 and delivery wire 100 (as described herein).

Once the delivery catheter 202 is inserted through an occlusion, such as a thrombus, the distal body 104 can be released from inside the delivery catheter 202 as described herein, and expanded.

Following removal of the delivery catheter 202, suction can be applied to the thrombus 212 or other blockage.

According to the invention, a suction step, as described herein, can be applied to any embodiment of a retrieval apparatus, and can be applied through the guide catheter, access catheter, specialized suction catheter, or some other type of catheter.

By way of example, a Seldinger technique can be initiated using a large bore suction catheter that is advanced over the delivery wire 100 (or a guide wire) and positioned on a proximal side of the thrombus 212, with the distal body 104 positioned on a distal side of the thrombus. Suction can then be applied to remove all or a portion of the thrombus.

The positioning of the distal body 104 on the distal side of the thrombus 212 can be used to retract the thrombus 212 in the direction of the suction device, thereby increasing the effectiveness of the suction device in removing the thrombus 212.

The distal body 104 also provides distal protection from distal embolization during the suction device's placement and/or during the suctioning procedure.

Note that in the above example, a proximal body, such as proximal body 102, has not yet been included in the procedure.

There are situations and, thus, embodiments where an optional proximal body 102 can be added to the procedure, for example, by slidably mounting a proximal body 102 to the delivery wire 100. As such, in some embodiments, the inclusion of a proximal body 102 is optional.

In some clinical scenarios, the suction procedure may result in only a partial removal of the thrombus 212 or other obstruction. In such scenarios, mechanical removal of the thrombus 212, using a distal body 104 and an added proximal body 102, may be advantageous and/or required.

Following the application of suction within the guide catheter 700, a proximal body 102 can be added to the delivery wire 100, where the proximal body 102 is disposed on a proximal side of the thrombus 212 or other obstruction. Once the proximal body 102 is placed on the delivery wire 100, it can be advanced towards the distal end of the delivery wire 100 by advancing the delivery wire 100.

In another example, the restrained proximal body 102 can be advanced towards the distal end of the delivery wire 100 using a hypo tube that is placed within the delivery catheter 202 over the delivery wire 100. As the hypo tube is pushed towards the distal end of the delivery wire 100, the restrained proximal body 102 can be moved axially to a desired location.

Once the restrained proximal body 102 is in the desired physical position relative to the thrombus 212 or other obstruction, the proximal body 102 can be released from inside the delivery catheter 202 and transition to an expanded configuration, as described herein.

The coaxially placed hypo tube can be pushed forwards and used to physically advance the expanded proximal body 102 to ultimately capture and compress the thrombus 212.

Once the thrombus 212 is captured/compressed between the proximal and distal bodies 102, 104, the entire retrieval device can be removed from the body via coaxially placed catheters/tubes, thus, permitting removal of the thrombus 212 from its prior resting place within the vessel 200.

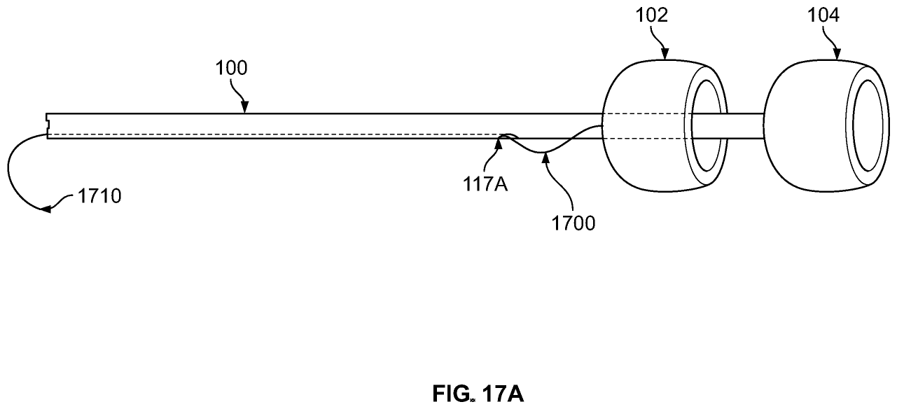

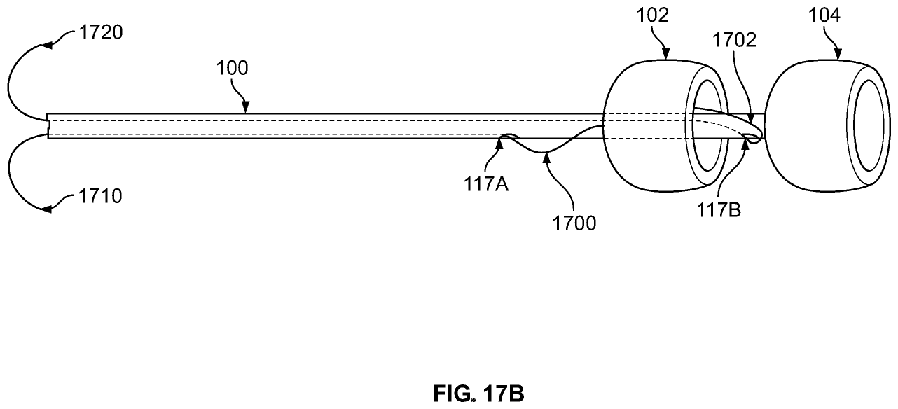

Referring now to FIGS. 17A and 17B, the proximal body 102 can be moved along the delivery wire 100 via application of force to a tether or multiple tethers. As shown in FIG. 17A, a single proximal tether 1700 can be attached to the proximal body 102; the proximal body 102 being slidably mounted and in releasable engagement with a delivery wire (or hypo tube), as described herein.

According to the invention, the proximal tether 1700 can be pulled to move the proximal body 102 back, proximally along the wire 100 after the proximal body 102 has been released and positioned in the manner described herein. The proximal tether 1700 can run parallel and within the delivery catheter (not shown) or, as shown in FIG. 17A, the proximal tether 1700 can run within a hollowed-out portion of the delivery wire 100 (or hypo tube) and emerge through an opening 117A.

FIG. 17B depicts a two-tether embodiment. As with the single-tether embodiment depicted in FIG. 17A, the tethers may run parallel and within the delivery catheter (not shown) or, as shown in FIG. 17B, the proximal tether 1700 and distal tether 1702 can run within a hollowed-out portion of the delivery wire 100 (or hypo tube) and emerge through openings 117A and 117B, where the proximal tether 1700 emerges from opening 117A and the distal tether 1702 emerges from opening 117B.

Movement of the proximal body 102 via the proximal tether 1700, in the proximal direction, is the same as mentioned above. In this embodiment, the interventionist can pull the end of the distal tether 1720 to move the proximal body 102 adjacent to the opening 117B, which results in a distal movement of the proximal body 102 without the need for distal movement via the delivery catheter as described herein.

In addition to the steps of deployment mentioned above, the following steps can also or alternatively be followed for using a retrieval device of the invention.

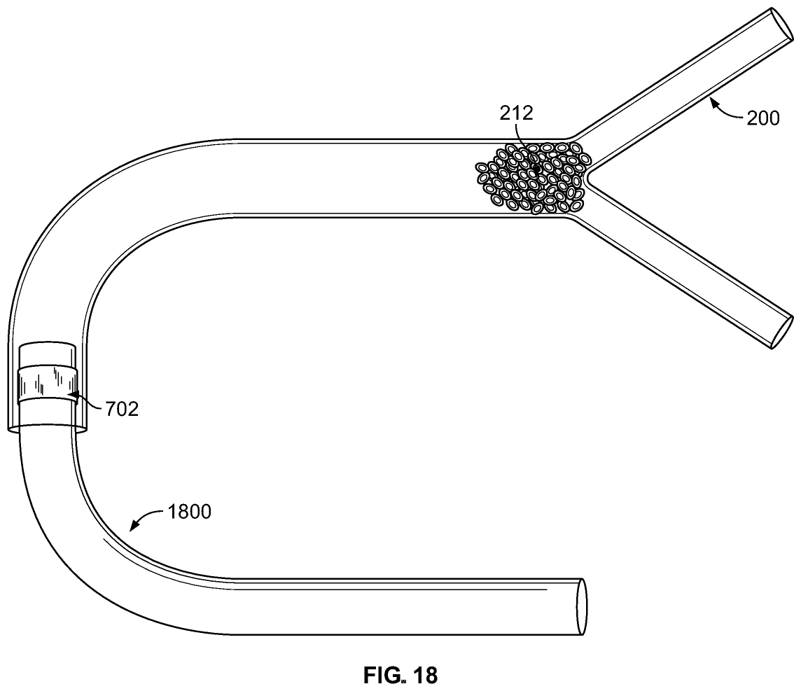

FIG. 18 depicts a vessel 200, which in this instance comprises a common carotid artery (CCA), having an occlusion therein 212 and a guide catheter 1800 having a deflated flow arrest balloon 702 inserted into the CCA.

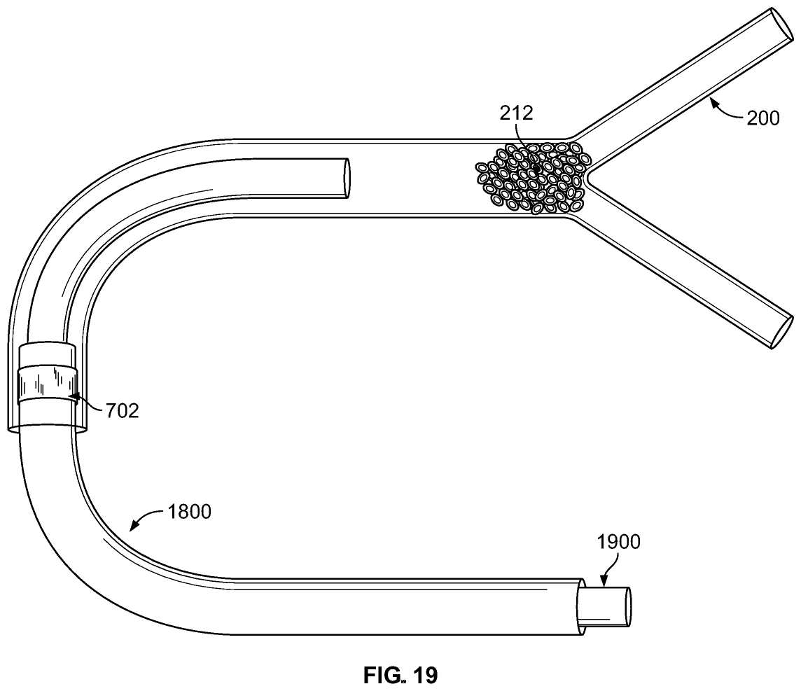

FIG. 19 depicts advancement of an access catheter 1900 distally through the guide catheter 1800 (in some embodiments, over a guide wire (not shown)).

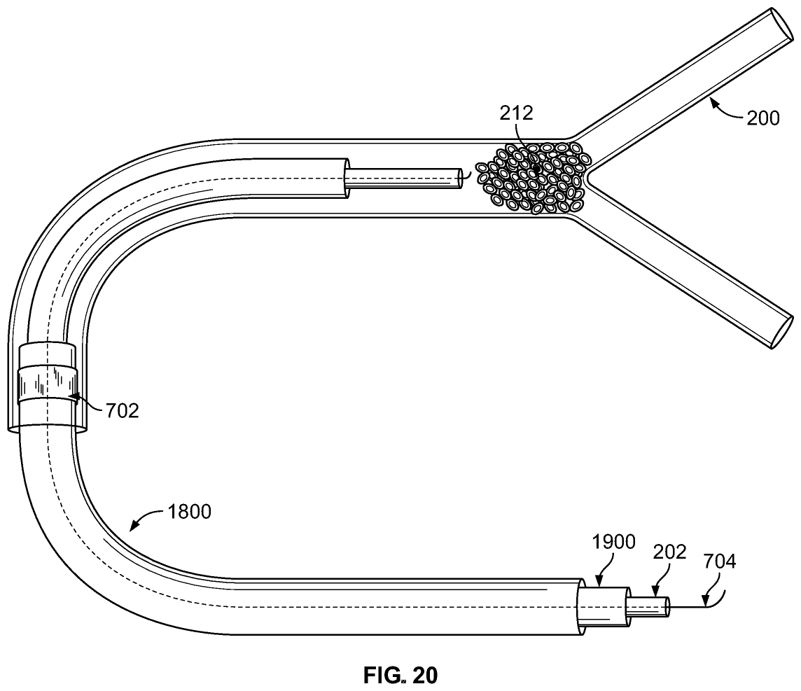

FIG. 20 depicts the distal advancement of a delivery catheter 202 via a guidewire 704 to the origin of the inclusion (i.e., the base of the thrombus 212).

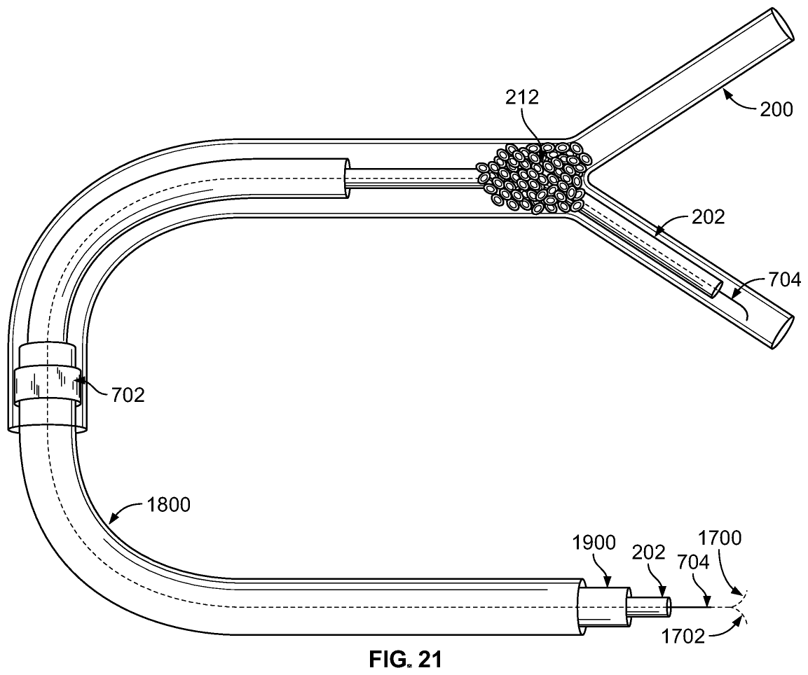

FIG. 21 depicts the flow arrest balloon 702 being temporarily inflated to arrest anterograde flow in the CCA and the delivery catheter 202 being advanced distal to the occlusion 212 via the guide wire 704 and, in this case, in M1.

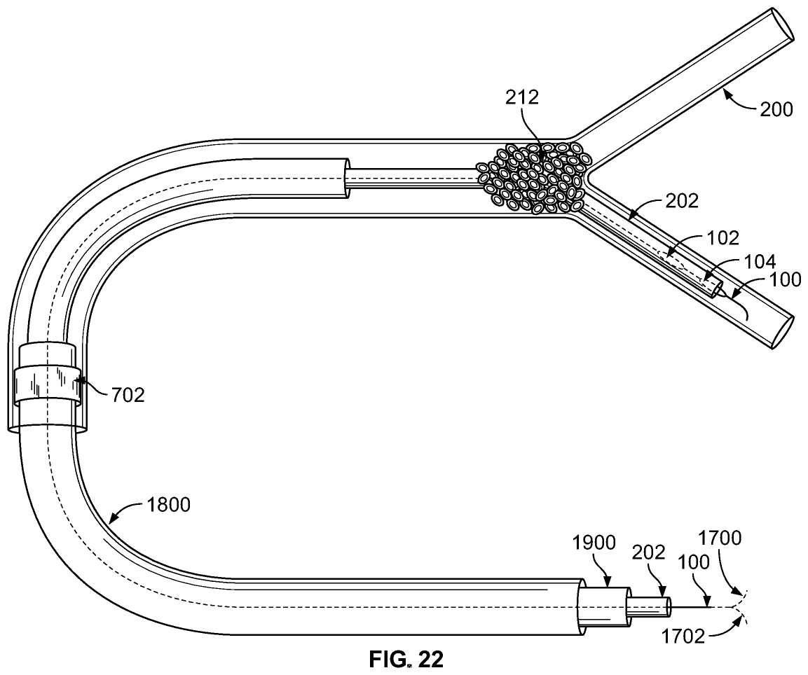

FIG. 22 depicts the delivery wire 100 with restrained proximal and distal bodies 102, 104 thereon inserted into and through the delivery catheter 202 with the tip emerging the delivery catheter. In this example, the delivery wire 100 has within it a proximal tether 1700 and a distal tether 1702.

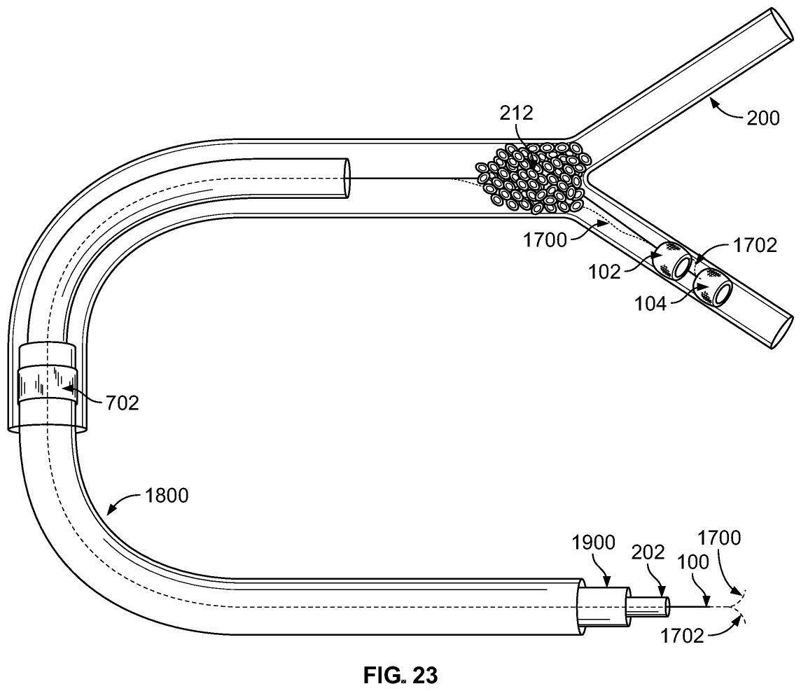

FIG. 23 depicts the expanded proximal 102 and distal 104 bodies deployed distally to the occlusion 212, and the proximal tether 1700 and the distal tether 1702, as described herein, attached to the proximal body 102.

FIG. 23 further depicts the deflation of flow arrest balloon 702 on the guide catheter 1800 to end flow arrest. Due to mesh structure of the proximal and distal bodies 102, 104, which are now deployed, anterograde flow into vessels can be re-established with protection (established via the expanded proximal and distal bodies 102, 104) from distal embolization of occlusion when flow is re-established. Suction can then be applied to the access catheter 1900 at this point.

FIG. 23 also depicts the delivery catheter 202 withdrawn to increase open luminal area in access catheter 1900, which allows for better suction when suction is applied to access catheter 1900.

With both the expanded proximal and distal bodies 102, 104 providing protection (most commonly initially in an M2 branch for an M1 occlusion or covering the M1 bifurcation for an ICA terminus) an interventionist can slowly pull the delivery wire 100 in a proximal direction. This will draw both bodies 102, 104 proximally (see FIGS. 23-25).

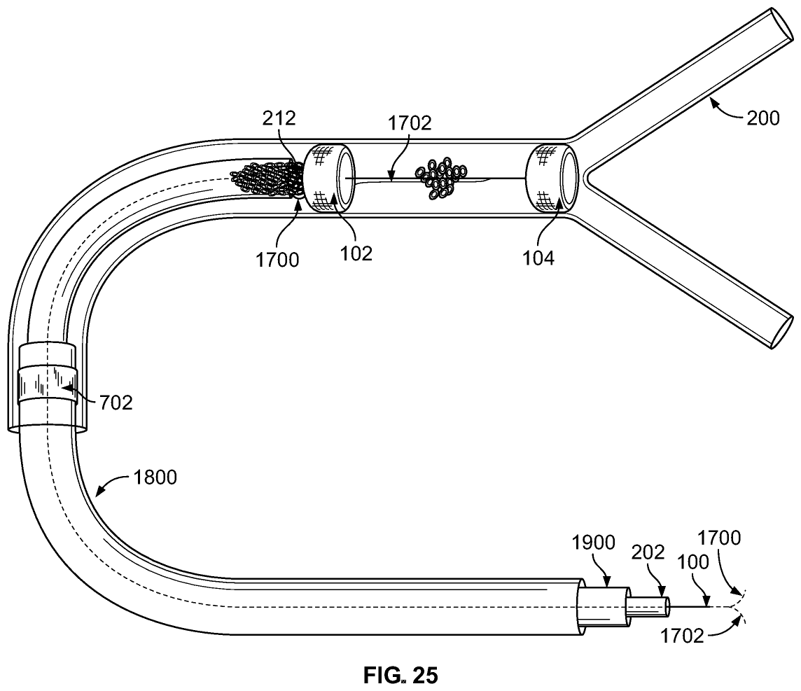

According to the invention, the proximal and distal bodies 102, 104 will open to a larger diameter when they transition from M1 to M2 and in the process of being withdrawn proximally will begin the thrombectomy process (see FIG. 25).

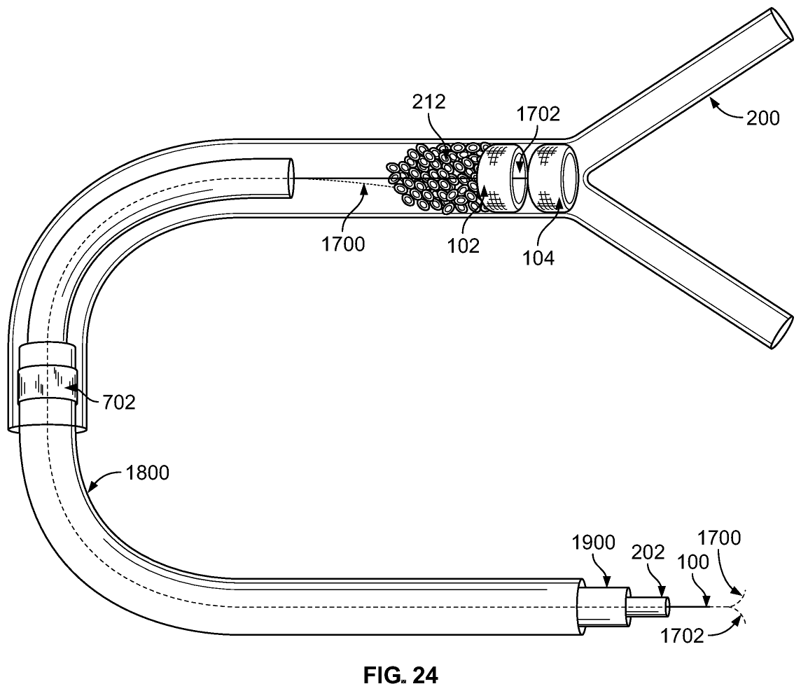

Once the distal body 104 opens at the M1 bifurcation, both superior and inferior M2 protection has been established (see FIGS. 24 and 25).

Using a proximal tether 1700 and a distal tether 1702 that connect to the proximal body 102 and exit from the delivery wire 100 either via an opening in the outer surface or via the opening on the end of the delivery wire 100 (see FIGS. 17A and 17B), the proximal body 102 can be moved along the M1, and possibly ICA lumen, back and forth (i.e., proximally and distally axially along the delivery wire) by pulling the ends of the tethers 1710, 1720, as described herein, and as desired by the clinician to mobilize the occlusion 212, loosen, and draw it proximally towards the suction device.

If the initial placement of the proximal body 102 is determined to be too far in the distal direction, the interventionist can use the proximal tether 1700 that is attached to the proximal body 102 to pull the proximal body 102 back in the proximal direction to place it farther from the distal end of the retrieval device. This process allows the interventionist to adjust the proximal body's position along the wire 100 instead of only being able to advance the proximal body 102 in the distal direction.

By way of example, the proximal body 102 can have a Kevlar tether that exits the delivery wire (or hypo tube) 100 at an opening distance about 1.0-2.0 cm proximal to the proximal side of the proximal body 102 to which it is attached. Therefore, while the two bodies 102, 104 are initially adjacent to each other, the proximal body 102, once electrolytically detached, can be withdrawn a distance proximally along the delivery wire 100 axis 1.0-1.5 cm by pulling on the proximal tether 1700.

The proximal body 102 can be advanced by pushing it forward with the delivery catheter 202 and/or a second, distal tether 1702 can exit the wire at opening 117B distally to the proximal body 102, which, when pulled, can pull the proximal body 102 distally back towards the distal body 104 and adjacent to the opening 117B. Therefore, by pulling proximal tether 1700 and/or the distal tether 1702 the proximal body 102 can slide backwards and forwards along the delivery wire 100. In this example, the noted configuration provides the proximal body 102 with 1.0-1.5 cm of travel distance back and forth along the delivery wire 100.

According to the invention, despite anterograde flow, the distal body 104 will provide protection against distal embolization of loosened/floating occlusion thus eliminating/reducing the risk of distal embolization of this material (see FIG. 25).

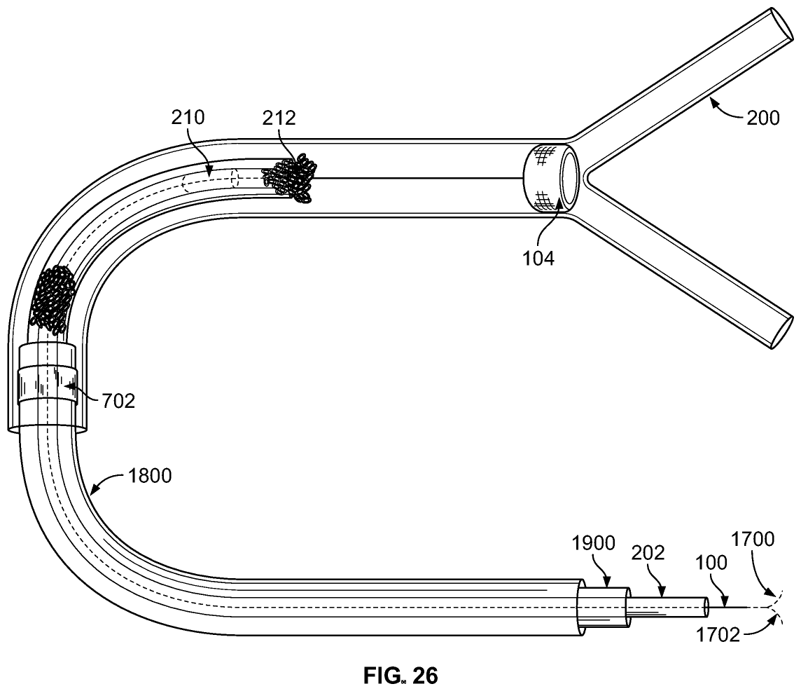

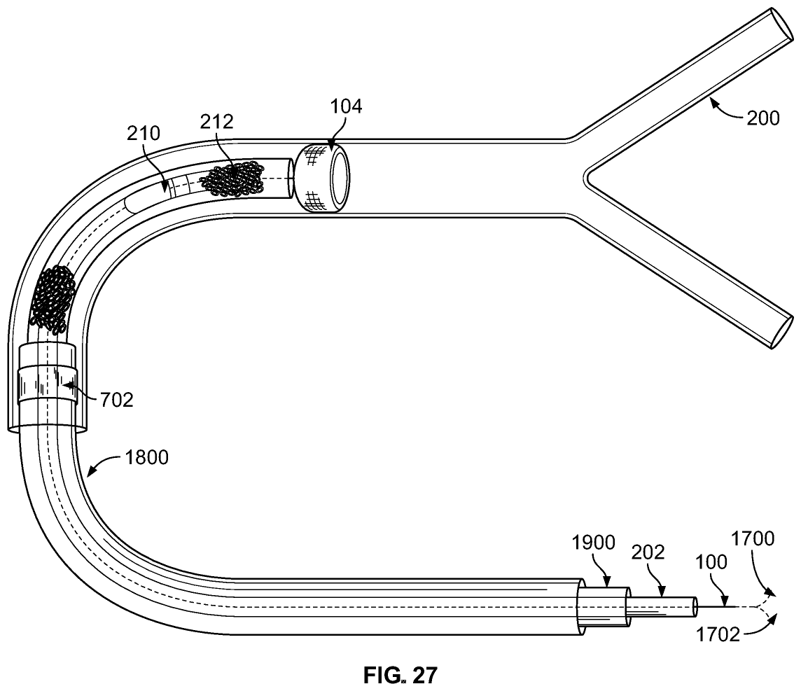

Once the thrombus 212 has been removed/evacuated through the access catheter 1900, the proximal and distal bodies 102, 104 can be removed by withdrawing them through the delivery catheter 202. This process will also mechanically draw any portion(s) of the thrombus 212 that sits on the tip of the access catheter 1900 (cleans the catheter tip) into the catheter 1900, so that it does not embolize off the catheter tip and back into the intracranial circulation (see FIGS. 25-27).

In some embodiments, the following steps are followed for using the retrieval device, as described herein, for capture of a foreign body (e.g., lost coil or fractured filter) and extraction intervention, such as an aneurysm coil lodged at an M1 bifurcation, wherein guide catheter 1800 is already in ICA from coiling procedure.

The delivery catheter 202 is initially advanced distal to the foreign body.

After the delivery catheter 202 is advanced distal to the foreign body, the retrieval apparatus is then advanced through the delivery catheter 202 (the “retrieval device”, again is the delivery wire 100 with the proximal and distal bodies 102, 104 mounted thereon in a manner described in this disclosure).

When the distal body 104 reaches the end of the delivery catheter 202, the interventionist can optionally detach the releasable engagement of proximal body 102, so it is free to move axially along the delivery wire 100.

After the retrieval device is advanced through the delivery catheter 202, the proximal tether 1700 is pulled, wherein the proximal body 102 is drawn proximally in the delivery catheter 202 so that there is a space, in some embodiments, a 1.0 cm space, between the two restrained proximal and distal bodies 102, 104 within the delivery catheter 202.

After the proximal tether 1700 is pulled, the delivery catheter 202 is retracted proximally, which allows allow the distal body 104 to open in the vessel 200 distal to the foreign body.

After the delivery catheter 202 is retracted proximally, the wire 100 is held in place while the delivery catheter 202 is drawn proximally until the proximal body 102 is unsheathed, wherein the proximal body 102 opens proximal to the foreign body/coil.

While holding the distal body 104 in place by holding the delivery wire 100 in place, the proximal body 102 can now be approximated along the wire 100 to the distal body 104 by either using the tethers 1700, 1702 as described herein, by simply pushing it forward with the delivery catheter 202, or by drawing the distal body 104 proximally.

After approximating the proximal body 102 along the delivery wire 100, the foreign body/coil is now trapped between the proximal and distal bodies 102, 104 and can be removed from the vessel 200.

As indicated above, the retrieval systems and apparatus of the invention can also be employed in non-vascular structures, such as bronchial pathways, and any other accessible space that contains a material (biologic or foreign) that necessitates removal or retrieval.

The retrieval systems and apparatus of the invention can also be employed to perform an intervention procedure to resolve, treat, or address a deep vein thrombosis (DVT) or a pulmonary embolism or perform any type of thrombectomy.

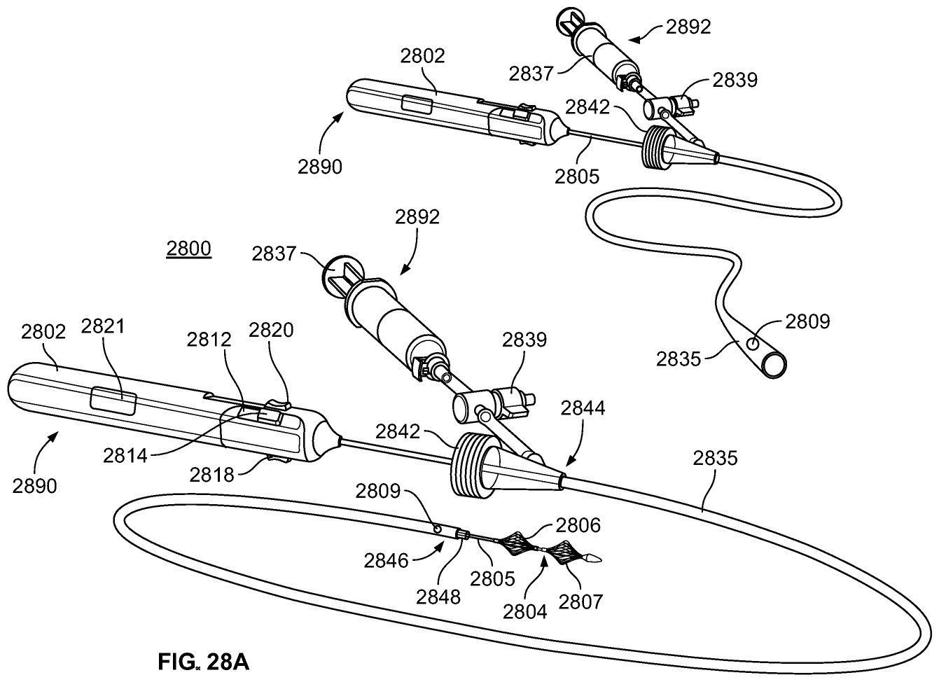

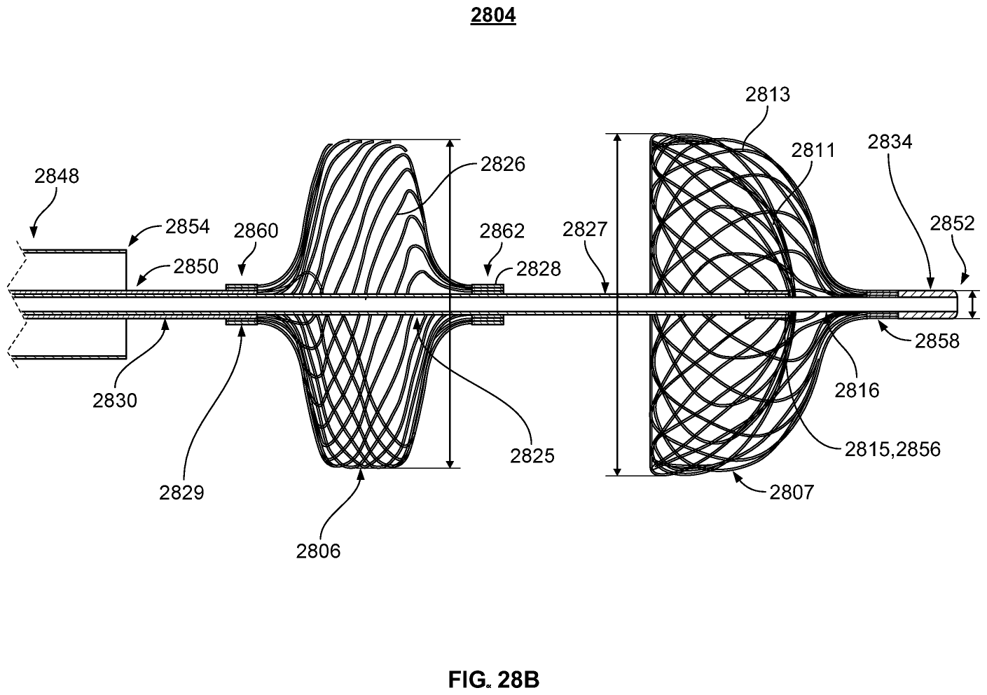

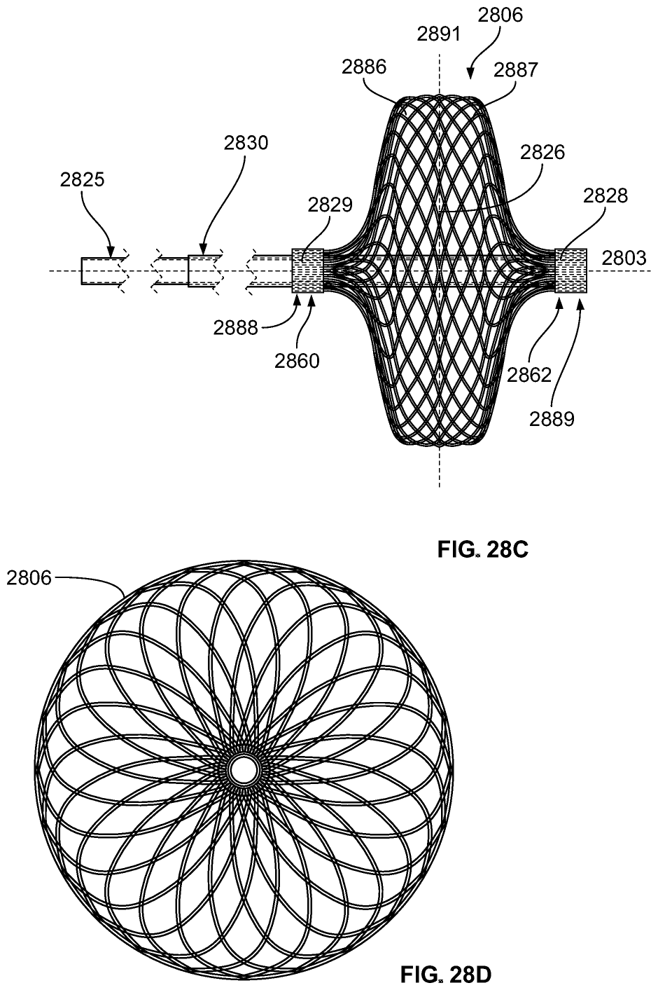

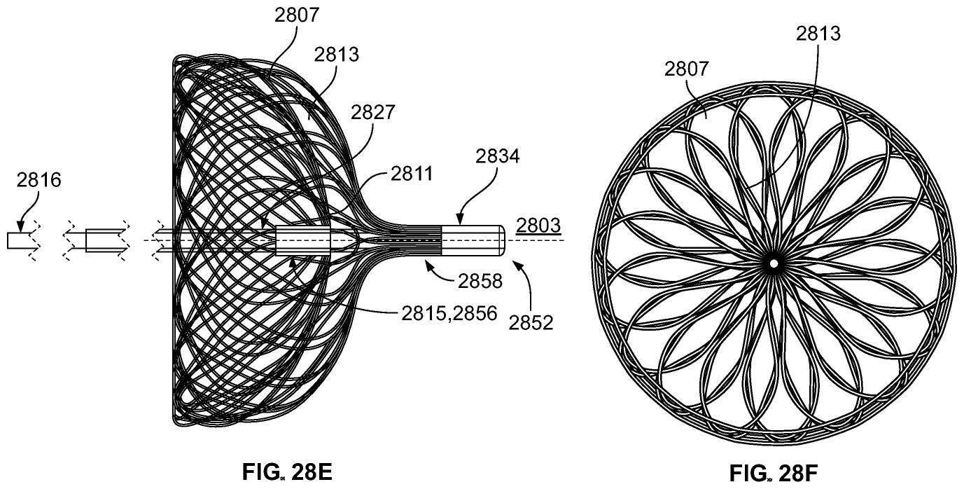

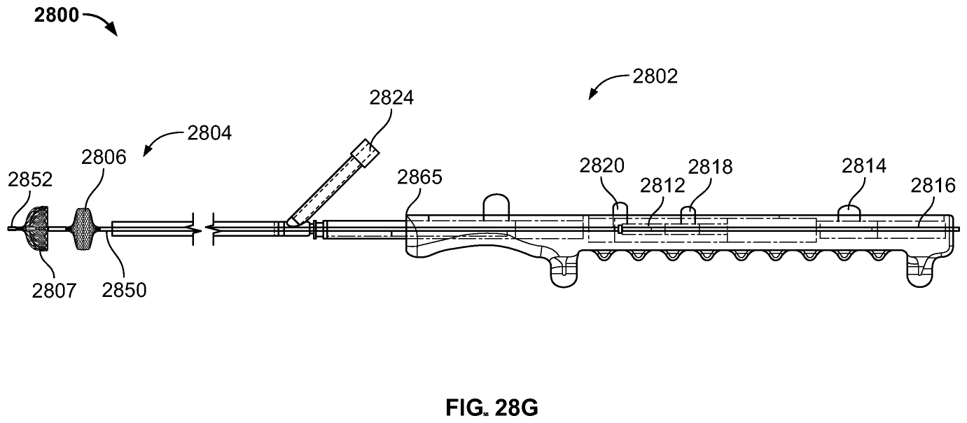

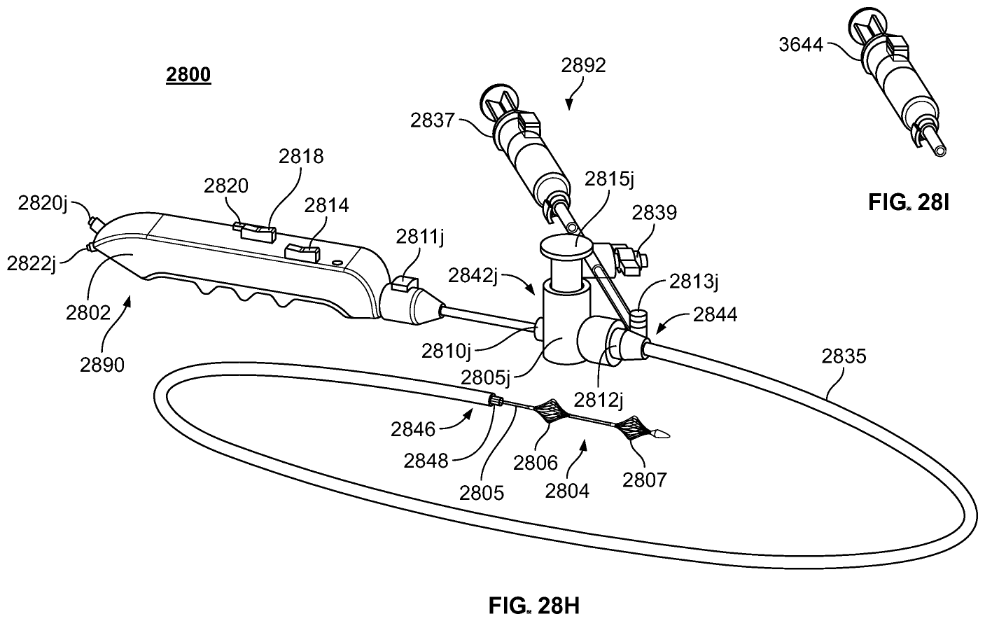

Referring now to FIG. 28A through 28G, there is depicted one embodiment of a retrieval apparatus, wherein:
a) FIG. 28A depicts a perspective view of the retrieval apparatus;
b) FIG. 28B depicts an expanded view of a distal tip portion of the retrieval apparatus shown in FIG. 28A;
c) FIG. 28C depicts a proximal element of the retrieval apparatus shown in FIG. 28A;
d) FIG. 28D depicts a front/top view of the proximal element shown in FIG. 28C, in an expanded state;
e) FIG. 28E depicts a mechanically expanded distal element of the retrieval apparatus shown in FIG. 28A;
f) FIG. 28F depicts a front/top view of the distal element shown in FIG. 28E in an expanded state; and
g) FIG. 28G depicts a plan view of the retrieval apparatus shown in FIG. 28A.

As depicted in FIG. 28A, retrieval apparatus 2800 comprises a first unit 2890 that includes a handle 2802 coupled to a proximal end of an elongated member 2805 having a plurality of telescoping tubes, such as at least four telescoping tubes, wherein a distal end of the elongated member 2805 has a tip portion 2804. The handle 2802 is configured to steer the tip portion 2804 in proximity to an occlusion.

The apparatus 2800 further comprises a second unit 2892 that includes an aspiration catheter 2835 having a suction source, such as, for example, a syringe 2837, a one-way valve 2839 and a port 2842; the port 2842 being coupled to a proximal end 2844 of the aspiration catheter 2835.

In some embodiments, the one-way valve 2839 is configured to direct suction through the aspiration catheter 2835.

For use during a procedure, the tip portion 2804 is placed into a delivery catheter 2848 and thereafter the delivery catheter 2848 is inserted into the aspiration catheter 2835, and follows through to port 2842, so that at least the tip portion 2804 projects distally from a distal end 2846 of the aspiration catheter 2835.

In some embodiments, as shown in the retrieval apparatus depicted in FIG. 28H, instead of port 2842, the apparatus 2800 includes a hemostasis valve hub 2842j. The valve hub 2842j is defined by an enclosure 2805j, a first opening 2810j in a first end of the enclosure 2805j, a second opening 2812j in a second end of the enclosure 2805j, and an actuator 2815j extending upward out of the enclosure 2805j.

In some embodiments, the proximal end 2844 of the aspiration catheter 2835 is coupled to the second opening 2812j.

According to the invention, when the actuator 2815j is depressed, the valve hub 2842j receives the elongated member 2805 through the first opening 2810j and allows the elongated member 2805 to pass through the second opening 2812j and through the aspiration catheter 2835.

In some embodiments, when the actuator 2815j is not depressed, the valve hub 2842j is configured to create a seal around a surface of the elongated member 2805.

In some embodiments, the suction source 2837 is coupled to a portion of the valve hub 2842j and is in pressure communication with the aspiration catheter 2835.

In accordance with aspects of the present specification, the retrieval apparatus 2800 is configured to enable an operator to single-handedly operate/actuate the handle portion 2802 (using first, second and third physically manipulable interfaces such as, by way of example, knobs, sliders, buttons or other actuation mechanisms 2814, 2818 and 2820) in order to mechanically expand, contract, or move a proximal member 2806 and/or a distal member 2807, as further discussed below.

In some embodiments, a first slider, knob, button, or other actuation mechanism 2814 is configured to mechanically expand or mechanically contract the proximal member 2806, a second slider, knob, button, or other actuation mechanism 2818 is configured to mechanically expand or mechanically contract the distal member 2807, and a third slider, knob, button, or other actuation mechanism 2820 is configured to axially move the proximal member 2806 relative to the distal member 2807, to axially move the proximal member 2806 while maintaining the distal member 2807 stationary, or to axially move the distal member 2807 while maintaining the proximal member 2806 stationary.

Preferably, the proximal and distal members 2806, 2807 disclosed herein are not self-expandable or self-contractable but, rather, only expand or contract when a pressure is manually applied or released using the physically manipulable interfaces (such as, for example, knobs, sliders, buttons or other actuation mechanisms) integrated into the handle 2802.

In some embodiments, as depicted in FIG. 28A, the first slider, knob, button, or other actuation mechanism 2814, the second slider, knob, button, or other actuation mechanism 2818, and the third slider, knob, button, or other actuation mechanism 2820 are positioned in an arc around an external surface of the handle 2802 such that each of the first, second, and third actuation mechanisms are at the same location, or within 3 inches, axially along the length of the handle.

In another embodiment, as depicted in FIG. 28H, the first slider, knob, button, or other actuation mechanism 2814, the second slider, knob, button, or other actuation mechanism 2818, and the third slider, knob, button, or other actuation mechanism 2820 are positioned on a flat external surface of the handle 2802 such that each of the first, second, and third actuation mechanisms are positioned adjacent to each other.

In some embodiments, as depicted in FIG. 28H, a proximal end of the handle 2802 includes a first guidewire port 2820j and a second flush port 2822j.

Also, in some embodiments, as depicted in FIG. 28H, a distal end of the handle 2802 includes a fourth physically manipulable interface, such as, for example, a slider, knob, button, or other actuation mechanism 2811j used to slidably advance or retract the delivery catheter 2848. The physically manipulable interface 2811j allows for a greater length of the delivery catheter 2848 to be passed through the hemostasis valve hub 2842j.

In some embodiments, pressing the physically manipulable interface 2811j allows the user to move or slide the physically manipulable interface 2811j towards or away from the tip portion 2804 thereby increasing or decreasing the length of the delivery catheter 2848 passing through the valve hub 2842j.

Further, in some embodiments, as depicted in FIG. 28H, the proximal end 2844 of the aspiration catheter 2835 (or the distal end of the valve hub 2842j) includes a port 2813j for injecting saline or a contrasting agent, when required.

In some embodiments, the handle comprises one or more actuation mechanisms to deliver pharmacological agents, such as a tissue Plasminogen Activator (tPA), and/or activate suction or aspiration while providing distal embolic protection.

In some embodiments, a method of treatment includes infusing a pharmacological agent, such as a tPA, into at least one lumen positioned within the catheter.

Preferably, the infusion is performed at the outset of the pulmonary embolism or deep vein thrombosis treatment process, while the proximal and/or distal members are still housed within the catheter, thereby covering the unexpanded proximal and/or distal members in tPA.

Alternatively, the infusion is performed at the outset of the pulmonary embolism or deep vein thrombosis treatment process, while the proximal and/or distal members are still housed within the catheter, directed through the distal end of the catheter, and injected directly into the clot prior to inserting and expanding the proximal and/or distal members.

In some embodiments, the catheter, e.g., delivery catheter 2848, and handle, e.g., handle 2890, in combination, are configured to deliver ultrasonic energy to a clot in order to accelerate lytic dispersion, drive medications deeper into the clot, speed the breakdown of the clot, and/or degenerate or unwind the fibrin quicker.

In some embodiments, the delivery catheter 2848 comprises an ultrasonic core in parallel with the elongated wire extending axially through the catheter lumen. The ultrasonic core is in electrical communication with a control unit positioned external to the catheter 2848.

The proximal end of the handle 2890 preferably comprises one or more leads in electrical communication with the ultrasonic core that would extend outward from the handle 2890 and be configured to connect to the control unit.

During the pulmonary embolism or deep vein thrombosis treatment process, ultrasonic energy can be delivered via the ultrasonic core using the control unit, at the beginning of the treatment upon delivery of the pharmacological agents, as described above.

In some embodiments, an ultrasonic core energy generator runs through the center of the delivery catheter 2848. In some embodiments, the ultrasonic core energy generator includes a control unit configured to manage the generator. In some embodiments, the proximal end of the retrieval apparatus 2800 includes lead plug(s) in electrical communication with the control unit.

In accordance with some aspects of the present specification, the first and second units 2890, 2892 are manufactured as separate standalone units or devices. This is advantageous in that a physician may use the first unit 2890 with any third-party aspiration catheter.

In some embodiments, the aspiration catheter 2835 is available with a plurality of external diameters, such as, but not limited to, 12 Fr, 16 Fr, 20 Fr, and 24 Fr (where Fr represents French scale or gauge system).

In some embodiments, the syringe 2837 has an exemplary, non-limiting, volume of cubic centimeters.

In some embodiments, the aspiration catheter 2835 is steerable or deflectable using the handle 2802. Embodiments of the steerable catheter are described in co-pending U.S. application Ser. No. 17/809,531, which is referenced herein in its entirety, and can be applied to all of the embodiments of the aspiration catheter of the present specification and as described in U.S. application Ser. No. 17/809,531.

In some embodiments, handle 2802 is configured to steer the tip portion of catheter 2835. In such embodiments, catheter 2835 is made steerable by an axial pull wire (not shown) that attaches from handle 2802 to the distal tip of catheter 2835. The pull wire is attached to a point on the circumference of the distal end of catheter 2835 such that when force is exerted on the wire, the catheter 2835 deflects towards the side where the wire is attached. At the proximal end of catheter 2835, the wire is preferably attached to an actuator within handle 2802, wherein the actuator enables an operator to axially tension the wire.

In some embodiments, for use in treatment of pulmonary embolism, the length of the delivery catheter 2848 is in the range of approximately 80.0 cm to 160.0 cm, more, preferably, the length of the delivery catheter 2848 is approximately 120.0 cm.

According to the invention, for use in treatment of pulmonary embolism, the aspiration catheter 2835 can have different lengths for different external diameters.

By way of example, in some embodiments, an aspiration catheter of 16 Fr has a length in a range of approximately 70.0 cm to 160.0 cm, more preferably, a length of approximately 112.0 cm, an aspiration catheter of 20 Fr has a length in a range of approximately 60.0 cm to 150.0 cm, more preferably, a length of approximately 105.0 cm or 106.0 cm, and an aspiration catheter of 24 Fr has a length in a range of approximately 50.0 cm to 130.0 cm, more preferably, a length of approximately 90.0 cm.

Also, in some embodiments, for use in treatment of pulmonary embolism (PE), the aspiration catheter of 20 Fr has a distal end or tip with a customizable 270° bend, whereas the aspiration catheter of 24 Fr has a flexible or bendable distal end or tip.

In some embodiments, for use in treatment of pulmonary embolism (PE), the suction source 2837 is a syringe having a volume ranging from approximately 1.0 cc to 100.0 cc, more preferably, a volume of approximately 60.0 cc.

In some embodiments, for use in treatment of deep vein thrombosis (DVT) the length of the delivery catheter 2848 is in a range of approximately 40.0 cm to 120.0 cm, more preferably, a length of approximately 80.0 cm.

In some embodiments, for use in treatment of deep vein thrombosis, a length of a 16 Fr aspiration catheter 2835 is in a range of approximately 45.0 cm to 80.0 cm, more preferably, a length of approximately 65.0 cm.

In some embodiments, for use in treatment of deep vein thrombosis, the suction source 2837 is a syringe having a volume ranging from approximately 1.0 cc to 100.0 cc, more preferably, a volume of approximately 60.0 cc.

In some embodiments, for use in treatment of right heart/atrium, the 24 Fr aspiration catheter has a length of approximately 90.0 cm.

In some embodiments, for use in treatment of IVC/SVC (Inferior Vena Cava/Superior Vena Cava), the 24 Fr aspiration catheter has a length of approximately 90.0 cm.

In some embodiments, at least one pressure transducer or sensor 2809 (such as, for example, a fiber-optic pressure sensor, electro-mechanical pressure sensor and hydraulic pressure sensor) is positioned at a distal end of aspiration catheter 2835.

In some embodiments, at least one pressure transducer or sensor 2809 is in the form of an elongated member that is co-extruded into the aspiration catheter 2835, so that the elongated member runs along a full length of the aspiration catheter 2835.

In some embodiments, the pressure transducer or sensor 2809 is in electrical communication with electronic circuitry located in a handle 2802 of the first unit 2890.

In some embodiments, the handle 2802 includes a pressure display 2821.

In various embodiments, the pressure transducer or sensor 2809 is configured to sense a pressure change or drop and, in particular, provide the physician with an indication that, as the occlusion is removed, there is an associated change of pressure indicative of a right side drop in right heart pressure. A right side drop in right heart pressure indicates that a problematic occlusion is being successfully removed.

Referring again to FIGS. 28A through 28H, in some embodiments, tip portion 2804 of apparatus 2800 has a proximal end 2850 and a distal end 2852. During operation of the apparatus 2800, the tip portion 2804 is inserted into, for example, a blood vessel for removing an occlusion therein while the handle portion 2802 remains in an operator/user's hands.

During insertion of the retrieval apparatus 2800 into the blood vessel, the distal end 2852 of the tip portion 2804 enters the blood vessel first and is placed in close proximity to the occlusion within the blood vessel by using the handle 2802 to maneuver the insertion of the tip portion 2804 in a desired position in the blood vessel.

As depicted in FIGS. 28A and 28H, the tip portion 2804 of retrieval apparatus 2800 comprises a distal element or body 2807, which, in a preferred embodiment, is a mechanically expandable, rigid anchor fixedly attached proximate the distal end 2852 of the tip portion 2804, and a proximal element or body 2806, which, in some embodiments, comprises a mechanically expandable pusher ball that is slidably mounted proximate the proximal end 2850 of the tip portion 2804. The mechanical expansion is in contrast to a non-mechanical expansion occurring because a shape memory material is naturally configured to adopt a pre-defined shape without mechanical force requiring to be applied.

In some embodiments, the proximal element or body 2806 is configured as a mechanically expandable, rigid anchor fixedly attached proximate to the proximal end 2850 of the tip portion 2804 while the distal element or body 2807 is configured as a mechanically expandable pusher ball that is slidably or moveably mounted proximate the distal end 2852 of the tip portion 2804.

In various embodiments, the proximal and distal elements 2806, 2807 are substantially curved structures.

In some embodiments, each of the proximal and distal elements 2806, 2807 is a three-dimensional (3D) shape.

In some embodiments, the proximal and distal elements 2806, 2807 are independent of one another, yet mounted on a single delivery retrieval apparatus (or system) 2800.

In some embodiments, the proximal member 2806 and the distal member 2807 are braided structures made of interwoven wires such that each structure has a plurality of open areas (allowing egress from outside the member into the internal volume of the member) formed by the braid. The open areas, relative to the total surface area of the proximal or distal member 2806, 2807, are preferably in a range of 1% to 99% of the total surface area.

In some embodiments, the proximal member 2806 has a greater percentage of open surface area than the distal member 2807, thereby allowing the proximal member 2806 to capture more clot material and the distal member 2807 to function more as a barrier to material flowing away from the device.

According to the invention, the proximal member 2806 and/or distal member 2807 can comprise any shape, including linear, spherical, spheroid, elliptical, ellipsoid, conical, polygonal, cylindrical, stent, chalice cup, umbrella, concave structure, convex structure, half-sphere, sphere, windsock, dumbbell, star, polygon, lever, disc, or a combination of such shapes.

In one embodiment, as depicted in FIG. 28C, the proximal member 2806 is structurally shaped as a first funnel 2886 having a neck 2888 directed along an axis 2803 in a proximal direction and a second funnel 2887 having a neck 2889 directed along the axis 2803 in a distal direction wherein the cup edge of the first funnel 2886 and the second 2887 are attached (in the form of contiguous wires) across a center axis 2891.

Alternatively or additionally, in some embodiments, the distal member 2807 is also structurally shaped similar to the proximal member 2806 in terms of including a first funnel having a first neck directed along the axis 2803 in a proximal direction and a second funnel having a second neck directed along the axis 2803 in a distal direction wherein the cup edges of the first funnel and the second funnel are attached, optionally, in the form of contiguous wires, across a center axis).

In some embodiments, when one of or both of the proximal element 2806 and/or the distal element 2807 is/are mechanically expanded, a proximal portion and a distal portion of the respective element expands first, followed by a center portion.

In some embodiments, each of the respective proximal, distal and center portions of the proximal element 2806 and the distal element 2807 expand at different rates.

In some embodiments, the proximal element 2806 and the distal element 2807 are heterogeneous, having different characteristics including, without limitation, radial force (as described further below), shape, size (for example, thickness, diameter), pore size (for example, mesh pore size or open areas as described above), and external coating.

In some embodiments, the proximal and distal elements 2806, 2807 are substantially similar in terms of the compositions and characteristics.

In some embodiments, the proximal and distal elements 2806, 2807 have similar braided structures that transition from a substantially linear structure to a substantially disc structure, adopting one or more three dimensional geometric shapes (spherical, spheroid, elliptical, ellipsoid, conical, polygonal, cylindrical, stent, chalice cup, umbrella, concave structure, convex structure, half-sphere, sphere, windsock, dumbbell, star, polygon, lever, disk or a combination of such shapes) during the transition. That is, the proximal element 2806 is defined by a first braid structure while the distal element 2807 is defined by a second braid structure, wherein the second braid structure is equivalent to the first braid structure, in embodiments.

In some embodiments, the second braid structure is not equivalent to the first braid structure.

As depicted in FIG. 28L of priority U.S. App. No. 63/368,325, in some embodiments, the proximal portion 2802I has a denser braid relative to the distal portion 2804I. That is, the proximal portion 2802I is defined by a braid or weave pattern that is more stiff, rigid, or dense as compared to the distal portion 2804I.

In some embodiments, the proximal element 2806 has a braid or weave that results in an active radial expansion.

In some embodiments, the proximal element 2806 is defined by a first braid structure while the distal element 2807 is defined by a second braid structure, wherein the second braid structure is equivalent to the first braid structure rotated 180 degrees.

According to the invention, the different braid structures enable scraping of unwanted material or occlusion from a vessel wall as well as effective trapping of the unwanted material or an occlusion within and/or between the proximal and distal elements 2806, 2807 so that the material or occlusion is easily removed.

According to the invention, the tip portion 2804 of the retrieval apparatus 2800 is at least partially enclosed within the delivery catheter 2848 which, when retracted, exposes at least the distal element 2807 and the proximal element 2806 when the apparatus 2800 is inserted and maneuvered within the vascular system or non-vascular structures, by using the handle portion 2802.

In some embodiments, the elongated member 2805, including the tip portion 2804, comprises four flexible telescoping tubes, which, when manipulated together, enable an operator/doctor to expand or contract the distal and proximal elements 2807, 2806 and move the proximal element 2806 axially, relative to the distal element 2807 and vice versa, in order to dislodge and remove the occlusion.

In some embodiments, the proximal element 2806 is configured to move relative to the distal element 2807 via manipulation of the flexible telescoping tubes to enable dislodging and removal of the occlusion.

In some embodiments, the distal and/or proximal elements 2807, 2806 are fabricated from a Nitinol™ wire mesh having a plurality of mesh pores, lattices, or cells.

In some embodiments, the distal and/or proximal elements 2807, 2806 are inflatable devices including, but not limited to, inflatable balloons.

In some embodiments, the distal and proximal elements 2807, 2806 are fabricated from different materials.

In some embodiments, the distal element 2807 comprises a wire mesh while the proximal element 2806 comprises an inflatable balloon.

In some embodiments, the distal element 2807 comprises an inflatable balloon while the proximal element 2806 comprises a wire mesh.

In some embodiments, mesh structures or patterns of the proximal and distal elements 2806, 2807 are configured such that fluids, including thrombolytic agents (e.g., tPA), may be infused into a vessel lumen in a controlled/controllable and selective manner.

In some embodiments, each of the proximal and distal elements 2806, 2807 have portions or sections of mesh wire with a diameter to allow infusion of fluids through the mesh wire pattern directly into the occlusion.

In some embodiments, the portions or sections of mesh wire include a plurality of pores to enable infusion of fluids therethrough.

Additionally, or alternatively, in some embodiments, one or more telescoping tubes located at the tip portion 2804 include a plurality of outlet ports for enabling fluids to be injected and sprayed therefrom.

In some embodiments, the outlet ports are positioned on a length of the tip portion 2804 extending from a proximal end 2860 of the proximal element 2806 to a distal end 2858 of the distal element 2807 (see FIG. 28B).

In some embodiments, each of the proximal element 2806 and the distal element 2807 are characterized by their ability to apply a variable radial force by virtue of the mechanical expansion being applied to each structure and the elements' stiffness or rigidity across sub-regions or portions of the respective elements 2806, 2807.

By way of example, in some embodiments, the expansion of each of the proximal and distal elements 2806, 2807 to a first size (defined by an area or volume encompassed by the element) may be characterized by a first radial force that first size can be applied to surrounding materials.

The expansion of each of the proximal and distal elements 2806, 2807 to a second size (defined by an area or volume encompassed by the element that is larger than the first size) may be characterized by a second radial force that second size can apply to surrounding materials, where the second radial force is different from the first radial force.

In some embodiments, each of the first and second radial forces are in a range of approximately 5.0 newtons to 25.0 newtons, more preferably, 10.0 newtons to 14.0 newtons.

The mechanical expansion allows for the intermittent, controlled expansion of the proximal and distal elements 2806, 2807 so that they can adopt and retain the shape of a first size (having a first area or volume), a second size (having a second area or volume), a third size (having a third area or volume), or a fourth size (having a fourth area or volume) under the control of the user and throughout the length of a procedure where the fourth size is bigger than the third size which is bigger than the second size which is bigger than the first size.

In some embodiments, each of the proximal element 2806 and the distal element 2807 are characterized by their ability to resist an application of a radial force, thereby maintaining its expanded shape, by virtue of the mechanical expansion being applied to each structure and the elements' stiffness or rigidity across sub-regions of the respective elements 2806, 2807.

By way of example, in some embodiments, the expansion of each of the proximal and distal elements 2806, 2807 to a first size (defined by an area or volume encompassed by the element) is characterized by an ability to resist (and therefore avoid collapse or compression of the first size) from a first radial force.

The expansion of each of the proximal and distal elements 2806, 2807 to a second size (defined by an area or volume encompassed by the element that is larger than the first size) can be characterized by an ability to resist (and therefore avoid collapse or compression of the second size) from a second radial force that is different from the first radial force.

In some embodiments, each of the first and second radial forces are in a range of approximately 5.0 newtons to 25.0 newtons, more preferably, approximately 9.0 newtons to 20.0 newtons, even more preferably, approximately 10.0 newtons to 14.0 newtons.

According to the invention, the mechanical expansion of the proximal and distal elements 2806, 2807 allows for the intermittent, controlled expansion of the elements 2806, 2807 so that they can adopt and retain the shape of a first size (having a first area or volume), a second size (having a second area or volume), a third size (having a third area or volume), or a fourth size (having a fourth area or volume) under the control of the user and throughout the length of a procedure where the fourth size is bigger than the third size which is bigger than the second size which is bigger than the first size.

It should further be appreciated that at least one of the proximal and distal elements 2806, 2807 are adapted to not collapse or compress when positioned against blood flow that applies a hydrostatic pressure in a range of approximately 80.0 mm Hg to 250.0 mm Hg. This is particularly valuable in arterial clot removal where the hydrostatic pressure level often causes other structures, particularly self-expanding structures, to compress or collapse.

According to the invention, a physician can use any of the embodiments disclosed herein by:
a) placing the distal element 2807 on a distal side of the occlusion;
b) placing the proximal element 2806 on a proximal side of the occlusion;
c) expanding each of the proximal and distal elements 2806, 2807 to a diameter, width, or volume that is greater than or equal to the diameter, width or volume of the vessel lumen it is positioned within of vessel (if greater than, it may be equal to or up to 150%, preferably around 110% to 130%, more preferably 120%);
d) sandwiching the thrombus between the distal and proximal elements 2806, 2807;
e) applying aspiration;
f) moving the proximal element 2806 to pull the thrombus to the catheter 2835;
g) partially collapsing the proximal and distal elements 2806, 2807 to move both toward the catheter 2835;
h) collapsing both elements 2806, 2807 to pull them back into the catheter 2835, with the thrombus; and
i) removing the catheter 2835 from the patient.

The distal element 2807 can optionally act as embolic protection to protect against occlusion, e.g., thrombus, material breaking free and flowing away from the procedure site.

In some embodiments, the proximal element 2806 and the distal element 2807 can have an anti-platelet coating to reduce adhesion and provide a less thrombogenic environment during clinical application.

In some embodiments, the proximal element 2806 and the distal element 2807 and any material (for example, wires and/or tubes) between the proximal and distal elements 2806, 2807 can be coated with control release agents including, but not limited to, thrombolytic agents.

Preferably, the distal element 2807 is rigid and holds/maintains a predefined shape after expansion.

In some embodiments of the invention, it is preferred that when deployed within a patient's vessel, a degree of fixation achieved by the distal element 2807 is greater than that achieved by the proximal element 2806, making the proximal element 2806 comparatively more mobile than the distal element 2807.

Having a distal member that is less susceptible to collapse and/or more resistant to force, ensures that the distal member can anchor and fix the apparatus within the vessel and provide the opposing leverage to ensure the proximal member, when axially moved, can pull the clot material toward the open catheter.

In alternate embodiments, however, the proximal element 2806 is rigid and holds/maintains a predefined shape after expansion while the distal element 2807 is relatively more mobile than the proximal element 2806. In such embodiments, the degree of fixation achieved by the proximal element 2806 is greater than that achieved by the distal element 2807.

Consequently, in such embodiments, the proximal element 2806 is less susceptible to collapse and/or more resistant to force, ensuring that the proximal element 2806 can anchor and fix the device within the vessel and provide the opposing leverage to ensure the distal element 2807, when axially moved, can pull the occlusion material toward the open catheter.

Fixation can be achieved by means of radial opposition against a patient's vessel wall, engagement within or beyond a distal portion of an occlusion requiring removal, deployment around distal anatomical features (such as a vascular bifurcation or curve in the vasculature), or any combination of these.

Aspects that may selectively enhance or impede the relative fixation enabled by the proximal and distal elements 2806, 2807 include radial force or stiffness, expanded diameter, braid density, braid wire size, deployed length, deployed shape, wire geometry and surface finish, surface treatments and coatings, or other means that allow for amplification or dampening of frictional engagement of the elements 2806, 2807 with the occlusion and surrounding vasculature.

In some embodiments, the proximal element 2806 is equally or less stiff/rigid than the distal element 2807 by a ratio of less than or equal to 1:10. In some embodiments, the relative stiffness or rigidity relationship is inverted with the distal element 2807 being equally or less stiff than the proximal element 2806 by a ratio of less than or equal to 1:10.

By modulating the manufactured relative radial force, stiffness or rigidity of the proximal and distal elements 2806, 2807, a desired balance of relative anchoring force to maceration potential can be achieved.

As previously stated, higher stiffness, rigidity or degree of fixation of the distal element 2807, relative to the proximal element 2806, provides an effective anchoring function when positioned within a vascular system or non-vascular structures.

In some embodiments, during a procedure, the anchored distal element 2807 provides an opposing anchoring force when the proximal element 2806, having relatively lesser stiffness, rigidity or degree of fixation, is moved or pushed axially towards the distal element 2807 to dislodge an occlusion. This provides an operator an improved ability to apply pressure, through the pushing manipulation of the proximal element 2806, in order to dislodge the occlusion.

Thus, in various embodiments, by adjusting the characteristics (of the proximal and distal elements 2806, 2807) such as the radial force, shape, size (for example, thickness, diameter), pore size (for example, mesh pore size in embodiments where at least one of the two elements 2806, 2807 is fabricated from a wire mesh) and external coating a desired stiffness, rigidity or flexibility of the retrieval apparatus 2800 may be obtained. With the desired stiffness, rigidity or flexibility the retrieval apparatus 2800 may be adapted to extract or remove a variety of obstructions such as, for example, stones, pulmonary emboli and deep vein clots.

In sum, the distal element 2807 has a higher degree of rigidity and greater degree of porosity as compared to the proximal element 2806. This is achieved by the distal element 2807 having at least one of a shape, wire thickness, average pore size, total porosity, total open surface area to total surface area ratio, and/or coating that is different from the proximal element 2806. As a result, when expanded to any number of a plurality of sizes, the distal element 2807 is preferably more resistant to changing its size or shape upon application of an external force as compared to the proximal element 2806.

In some embodiments, that external force is in a range of approximately 1.0 newton to 50.0 newtons and any increment therein, more preferably, approximately 8.0 newtons to 20.0 newtons, even more preferably, approximately 9.0 newtons to 15.0 newtons.

Additionally, as a result, when expanded to the same size as the proximal element 2806, the distal element 2807 is preferably less porous, meaning that it has less open surface area relative to total surface area, than the proximal element 2806.

As indicated above, in some embodiments, the elongated member 2805, including the tip portion 2804, comprises a plurality of telescoping tubes (also referred to, alternatively, as shafts), such as 1-6 or preferably 4, which facilitate axial movement of the proximal element 2806 and the relative distance between the proximal element 2806 and the distal element 2807.

As depicted in FIG. 28B, a first tube 2830 is shown projecting distally from a distal end 2854 of the delivery catheter 2848. The first tube 2830 is coupled with a second tube 2825 that is coupled with a third tube 2827 which, in turn, is coupled with a fourth/anchor tube 2816. The fourth/anchor tube 2816 forms the distal end 2852 of the tip portion 2804.

In some embodiments, the four tubes 2830, 2825, 2827 and 2816 are arranged as a coaxial array of telescopic tubes, all of which are designed to be able to move axially relative to one another.

In some embodiments, the first tube 2830 is concentrically positioned around the second tube 2825, the second tube 2825 is concentrically positioned around the third tube 2827, and the third tube 2827 is concentrically positioned around the fourth tube 2816.

In a preferred embodiment, the four telescoping tubes 2830, 2825, 2827 and 2816 can be axially expanded or contracted relative to each other by using the handle portion 2802.

In some embodiments, the telescoping tubes 2830, 2825, 2827 and 2816 comprise a shape-memory NiTi alloy, preferably, Nitinol™.

In some embodiments, the distal element 2807 has a proximal end 2856 and a distal end 2858.

As depicted in FIG. 28B, the distal end 2858 is fixedly connected on the fourth tube 2816 at the anchor nose 2834, while the proximal end 2856 is fixedly connected to a point 2815 on the third tube 2827 in both expanded and non-expanded states of the distal element 2807.

In some embodiments, the wire mesh 2813 is only attached at points 2834 and 2815, respectively, of an exterior surface of the fourth tube 2816 and the third tube 2827, while the remaining portion of the wire mesh 2813 is unattached and therefore free to expand or contract.

In some embodiments, in a non-expanded or collapsed state, the distal element 2807 (comprising a plurality of wires) forms a generally tubular wire mesh 2813 and is concentrically positioned around a lumen of the fourth tube 2816.

It should be appreciated that, while the term “tube” is used to describe the telescoping structures, any type of cylindrical, hollow wire, solid wire, or other elongated structure may be used and the term “tube” or “shaft” is just intended to cover each of these structures.

Upon axial compression of the third tube 2827 relative to the fourth tube 2816, the wire mesh 2813 expands radially around the lumen of the fourth tube 2816.

Similarly, upon axial decompression of the third tube 2827 relative to the fourth tube 2816, the wire mesh 2813 is induced to compress or contract radially around the lumen of the fourth tube 2816.

In some embodiments, in order to expand or contract the distal element 2807 the fourth tube 2816 is moved axially while the first, second and third tubes 2830, 2825, 2827 remain stationary.

In some embodiments, initial expansion of the distal element 2807, as induced by relative axial motion of the third tube 2827 and the fourth tube 2816, is such that the distal element 2807 first takes a shape similar to that of the proximal element 2806 (the shape of the proximal element 2806 being substantially elliptical, in one embodiment). Further relative axial movement of the third tube 2827 and the fourth tube 2816 induces an inversion in at least a portion of the wire mesh 2813 such that the proximal end 2856 collapses inside the distal end 2858, forming a chalice or a cup/concave shape, as depicted in FIGS. 28B and 28E.

Consequently, in some embodiments, the expanded distal element 2807 has a shape substantially equivalent to a semi-sphere or cone with an interior surface 2811 folded into the semi-sphere to form a chalice or cup-like structure which may be used to hold an occlusion before removal from a patient's body.

In some embodiments, a distance between the two attachment points 2834 and 2815 of the wire mesh 2813 ranges approximately from approximately 1.0 mm to 100.0 mm.

Stated differently, relative axial movement of the third tube 2827 and the fourth tube 2816 causes the proximal end 2856 and the distal end 2858 to move closer to each other, the material comprising the distal element 2807 and extending between the ends 2856 and 2858 is compressed and therefore expands outward.

In contrast, as the proximal end 2856 and the distal end 2858 move away from each other, the material comprising the distal element 2807 and extending between the ends 2856 and 2858 is stretched and therefore collapses down to, and elongates along, a body lumen. Thus, the distal element 2807 expands by having the proximal end 2856 move distally and contracts by having the proximal end 2856 move proximally relative to the distal end 2858.

In some embodiments, the anchor nose 2834 is configured as a corkscrew structure such that, as the tip portion 2804 is advanced towards a clot (within a vessel lumen) the tip portion 2804 is rotated to “screw into” and break up the clot material.

Referring now to FIGS. 28B, 28C and 28D, in some embodiments, the proximal element 2806 has a proximal end 2860 and a distal end 2862. The distal end 2862 of the proximal element 2806 is fixedly attached to the second tube 2825 at a point 2828, while the proximal end 2860 is fixedly attached to the first tube 2830 at a point 2829 in both expanded and non-expanded states of the proximal element 2806.

In some embodiments, in a non-expanded state, the proximal element 2806 (comprising a plurality of wires) forms a wire mesh 2826 concentrically positioned around a lumen of the second tube 2825.

In some embodiments, a portion of the wire mesh 2826 is only attached at points 2828 and 2829, of an exterior surface of the second tube 2825 and the first tube 2830, respectively, while the remaining portion of the wire mesh 2826 is unattached and therefore free to expand or contract.

Upon axial compression of the first tube 2830 relative to the second tube 2825, the wire mesh 2826 is induced to expand radially around the lumen of the second tube 2825.

Similarly, upon axial decompression of the first tube 2830 relative to the second tube 2825, the wire mesh 2826 is induced to compress or contract radially around the lumen of the second tube 2825.

In some embodiments, in order to expand or contract the proximal element 2806 the first tube 2830 is moved axially while the second, third, and fourth tubes 2825, 2827, 2816 remain stationary.

Stated differently, relative axial movement of the first tube 2830 and the second tube 2825 causes the proximal end 2860 and the distal end 2862 to move closer to each other, whereby the material comprising the proximal element 2806 and extending between the ends 2860 and 2862 is compressed and therefore expands outward.

In contrast, as the proximal end 2860 and the distal end 2862 move away from each other, the material comprising the proximal element 2806 and extending between the ends 2860 and 2862 is stretched and therefore collapses down to, and elongates along, a body lumen. Thus, the proximal element 2806 expands by having the proximal end 2860 move distally and contracts by having the proximal end 2860 move proximally relative to the distal end 2862.

In some embodiments, in an expanded state the proximal element 2806 approximates an elliptical shape wherein, at least a portion of the wire mesh 2826 lies approximately perpendicular to the lumen of the second tube 2825.

In some embodiments, a fully expanded proximal element 2806 is substantially elliptical or disc-shaped, as depicted in FIGS. 28C and 28D, while in a transient or less expanded state the proximal element 2806 can comprise different curved shapes, such as, for example, substantially spherical.

In some embodiments, a fully expanded proximal element 2806 is substantially spherical shaped, while in a transient or less expanded state the proximal element 2806 is substantially elliptical shaped.

As previously discussed, according to the invention, in an expanded state the proximal element 2806 can comprise the form of a cylinder, stent, chalice cup, umbrella, concave structure, half-sphere, sphere, windsock, dumbbell, star, polygon, lever, or any other suitable shape configured for aiding retrieval of the occlusion.

It should be appreciated that in an expanded state, in some embodiments, the proximal element 2806 can comprise a first shape while the distal element 2807 can comprise a second shape.

In some embodiments, the first shape is different from the second shape.

In some embodiments, the first and second shapes are substantially similar.

In some embodiments, the proximal and/or distal elements 2806, 2807 can be turned and rotated as motorized units.

In such an embodiment, a small motor positioned in or proximate the handle 2890 is coupled to each of the proximal and/or distal elements 2806, 2807 and, upon actuation, the motor causes one of or both the proximal and/or distal elements 2806, 2807 to move or rotate.

In some embodiments, the distance between the distal end 2865 of the handle portion 2802 and the distal end 2852 of the tip portion 2804 is in a range of approximately 0.5 mm to 110.0 cm.

Referring now to FIG. 28A, in a preferred embodiment, the handle 2890 comprises a first actuator, knob, slider or button 2814 that is configured to enable the user to mechanically expand or contract the distal element 2807, which is coupled to the third tube 2827 and is slidably fitted into the groove 2812 such that the first slider 2814 may be slid forward (that is, distally) towards the tip portion 2804 or backwards (that is, proximally) away from the tip portion 2804.

As depicted in FIG. 28B, the fourth tube 2816 is connected to the distal element 2807 at point 2834 while the third tube 2827 is connected to the distal element 2807 at point 2815. A sliding movement of the third tube 2827 relative to the fourth tube 2816 induces and aids in the expansion and contraction of the distal element 2807.

It should be appreciated that, in alternate embodiments, the first actuator, knob, slider or button 2814 is coupled to the fourth tube 2816 so that a sliding movement of the fourth tube 2827 (using the first actuator, knob, slider or button 2814) relative to the third tube 2827 induces and aids in the expansion and contraction of the distal element 2807. In other words, the first actuator, knob, slider or button 2814 can be coupled to either the third tube 2827 or the fourth tube 2816 in order to impart a sliding movement of the third and fourth tubes 2827, 2816 relative to each other.

Sliding the first knob 2814 within the groove 2812 towards the tip portion 2804 causes the third tube 2827 to telescope into the fourth tube 2816 thereby inducing an axial compression of the third tube 2827 relative to the fourth tube 2816 and consequently of the distal element 2807 between the proximal and distal ends 2856, 2858 of the wire mesh 2813. This causes the wire mesh 2813 (and therefore the distal element 2807) to expand radially around the lumen of the fourth tube 2816 and assume an expanded chalice, cup/concave shape having a diameter greater than a diameter in an unexpanded state. This kind of mechanical expansion of the distal element 2807 is preferred as this expansion provides a user control over the diameter of the distal element 2807 in an expanded state and, also provides a rigid structure that is less susceptible to collapse when placed under pressure.

Providing a physician control over the expansion size means that a physician can set one of a plurality of different expansion sizes and, upon setting that expansion size, the proximal or distal element maintains that expansion size even upon application of an external force in the ranges disclosed herein, such as 9.0 newtons to 15.0 newtons.

In some embodiments, a proximal element and/or distal element (first expansion member and/or second expansion member) 2806, 2807 expands upon moving the at least one actuation mechanism (slider, knob, lever, etc.) distally and wherein said expansion causes at least one of the first expandable member 2806 and the second expandable member 2807 to transform from a substantially linear configuration to a first shape, second shape or third shape depending on how far the at least one actuation mechanism has been moved distally.

The first shape, second shape or third shape is at least one of a spherical shape, an elliptical shape, a conical shape, a polygonal shape, a cylindrical shape, a shape of a stent, a shape of a chalice cup, a shape of an umbrella, a concave shape, a convex shape, a half-sphere shape, a windsock shape, a dumbbell shape, a star shape, or any combination of said shapes.

The first shape has a first outer surface and a furthest distance from the first outer surface to the elongated member is defined by a first distance; the second shape has a second outer surface and a furthest distance from the second outer surface to the elongated member is defined by a second distance; the third shape has a third outer surface and a furthest distance from the third outer surface to the elongated member is defined by a third distance and the third distance is greater than the second distance and wherein the second distance is greater than the first distance.

In some embodiments, the first shape is configured to maintain said first distance even upon an application of an external force to the first outer surface in a range of approximately 9.0 newtons to 15.0 newtons, wherein the second shape is configured to maintain said second distance even upon an application of an external force to the second outer surface in a range of approximately 9.0 newtons to 15.0 newtons, and wherein the third shape is configured to maintain said third distance even upon an application of an external force to the third outer surface in a range of approximately 9.0 newtons to 15.0 newtons.

Similarly, sliding the first knob 2814 within the groove 2812 away from the tip portion 2804 causes the third tube 2827 to telescope and expand out of the fourth tube 2816 thereby inducing an axial decompression of the distal element 2807 between the proximal and distal ends 2856, 2858 of the wire mesh 2813. This causes the wire mesh 2813 (and therefore the distal element 2807) to contract radially around the lumen of the fourth tube 2816 and assume an unexpanded substantially cylindrical shape having a diameter lesser than a diameter in an expanded state.

In some embodiments, the distal element 2807 assumes a substantially cylindrical shape when in a fully collapsed or unexpanded state, a substantially elliptical shape when in a partially expanded state and a concave, umbrella, half-sphere, sphere, windsock, dumbbell, star, polygon, chalice or cup-like shape when in a fully expanded state.

In some embodiments, the first knob 2814 locks (cannot be moved further forward) in a position in the groove 2812 when the distal element 2807 has expanded to a maximum diameter, which in an embodiment is approximately 25.0 mm. Thus, sliding the first knob 2814 forward enables the user to expand the distal element 2807 to a plurality of intermediate diameters and up to a maximum permissible diameter.

In some embodiments, the first knob 2814 is provided with a “clutch” feature so that, when opposing pressure is experienced from walls of a blood vessel during expansion of the distal element 2807, the “clutch” clicks in and, hence, provides an audio indication, so that the user does not over expand the distal element 2807. This feature is advantageous since it prevents the user from damaging the blood vessel due to over expansion of the distal element 2807.

According to the invention, the distal element 2807 can expand to a diameter depending upon an application/functional use of the retrieval apparatus 2800. By way of example, for use in treatment of pulmonary/large vessel having a diameter of up to approximately 20.0 mm, the diameter of an expanded distal element 2807 ranges from approximately 10.0 mm to 25.0 mm; for use in treatment of peripheral or deep vein thrombosis vessel having a diameter ranging from approximately 5.0 mm to 10.0 mm, the diameter of an expanded distal element 2807 ranges from approximately 3.0 mm to 12.0 mm; for use in treatment of neuro or brain vessels, the diameter of an expanded distal element 2807 ranges from approximately 2.0 mm to 10.0 mm; for use in retrieval of an occlusion in the inferior vena cava (IVC) vessels, the diameter of an expanded distal element 2807 ranges from approximately 35.0 mm to 40.0 mm.

In some embodiments, for use in treatment of pulmonary embolism, each of the proximal and distal elements 2806, 2807, in expanded form, has an outer diameter ranging from approximately 3.0 mm to 16.0 mm.

In some embodiments, for use in treatment of pulmonary embolism, each of the proximal and distal elements, in expanded form, has a maximum outer diameter of approximately 16.0 mm.

In some embodiments, for use in treatment of pulmonary embolism, the delivery catheter is approximately 9 Fr.

In some embodiments, for use in treatment of deep vein thrombosis, each of the proximal and distal elements 2806, 2807, in expanded form, has an outer diameter ranging from approximately 3.0 mm to 20.0 mm.

In some embodiments, for use in treatment of deep vein thrombosis, the proximal element 2806 has a maximum outer diameter of approximately 16.0 mm and the distal element 2807 has a maximum outer diameter of approximately 20.0 mm.

In some embodiments, for application of the retrieval apparatus 2800 in treatment of pulmonary embolism the proximal element 2806 is designed with a larger diameter as compared to the distal element 2807.

In some embodiments, if a diameter of the distal element 2807, in expanded form, is approximately 16.0 mm, the diameter of the proximal element 2806, in expanded form, is approximately 20.0 mm, as the internal diameter of a patient's blood vessels tapers down distally.

In some embodiments, if a diameter of the distal element 2807, in expanded form, is approximately 5.0 mm, then the diameter of the proximal element 2806, in expanded form, is approximately 20.0 mm.

In some embodiments, for application of the retrieval apparatus 2800 in treatment of deep vein thrombosis the proximal element 2806 is designed with a smaller diameter as compared to the distal element 2807.

In some embodiments, if a diameter of the distal element 2807, in expanded form, is approximately 16.0 mm, then the diameter of the proximal element 2806, in expanded form, is approximately 14.0 mm, as in this case, the internal diameter of a patient's blood vessels is larger proximally.

In another embodiment, if the diameter of the distal element 2807, in expanded form, is approximately 16.0 mm then the diameter of the proximal element 2806, in expanded form, is approximately 8.0 mm.

In some embodiments, in fully expanded state, each of the proximal and distal elements 2806, 2807 has a diameter ranging from approximately 5.0 mm to 30.0 mm, more preferably, 10.0 mm to 25.0 mm, even more preferably 10.0 mm to 20.0 mm.

In some embodiments, the groove 2812 contains a series of interlocking features along its length such that the first knob 2814 can be selectively engaged or disengaged from a locked position in the handle 2802 at a plurality of expanded diameters for the distal element 2807.

In some embodiments, the handle portion 2802 includes a second actuator, knob, slider or button 2818 configured to enable the user to mechanically expand or contract the proximal element 2806.

In some embodiments, the handle portion 2802 includes a third actuator, knob, slider or button 2820 configured to enable the user to mechanically slide the proximal element 2806 forward towards the distal element 2807 or backwards away from the distal element 2807 that remains stationary.

In some embodiments, the third actuator, knob, slider, or button 2820 is configured to enable the user to mechanically slide the distal element 2807 back towards the proximal element 2806 or forwards away from the proximal element 2806 that remains stationary. The second and third knobs 2818, 2820 are slidably fitted into the groove 2812.

In some embodiments, the second slider 2818 is coupled with the first tube 2830 to enable the user to mechanically expand or contract the proximal element 2806. Alternatively, the second slider 2818 may be coupled with the second tube 2825 to enable the user to mechanically expand or contract the proximal element 2806.

In some embodiments, the third slider 2820 is coupled with the first and second tubes 2830, 2825 such that a sliding movement of the third slider 2820 towards or away from the tip portion 2804 causes the proximal element 2806 to slide towards or away from the distal element 2807 (while the third and fourth tubes 2827, 2816 remain stationary).

In some embodiments, the third slider 2820 is coupled with the third and fourth tubes 2827, 2816 such that a sliding movement of the third slider 2820 towards or away from the tip portion 2804 causes the distal element 2807 to slide away from or towards the proximal element 2806 (while the first and second tubes 2830, 2825 remain stationary).

In some embodiments, the first tube 2830 is connected to the proximal element 2806 at the point 2829 while the second tube 2825 is connected to the proximal element 2806 at the point 2828. A sliding movement of the first tube 2830 relative to the second tube 2825 aids in the expansion and contraction of the proximal element 2806. Upon axial compression of the first tube 2830 relative to the second tube 2825, the wire mesh 2826 is induced to expand radially around the lumen of the second tube 2825.

In some embodiments, when the second slider 2818 is moved or slid in the groove 2812 towards the tip portion 2804, this causes the first tube 2830 to telescope into the second tube 2825, thereby inducing an axial compression of the first tube 2830 relative to the second tube 2825.

Consequently, the proximal element 2806 is caused to expand to a desired diameter. When the second slider 2818 is moved away from the tip portion 2804 the first tube 2830 is slid out of (or telescoped through) the second tube 2825 thereby inducing an axial decompression (or elongation) of the first tube 2830 relative to the second tube 2825 between the proximal and distal ends 2829, 2828 of the wire mesh 2826. This causes the wire mesh 2826 (and therefore the proximal element 2806) to contract radially around the lumen of the second tube 2825 and assume an unexpanded shape having a diameter lesser than a diameter in an expanded state or assume a fully unexpanded state.

In some embodiments, when the third slider 2820 is moved in the groove 2812 towards the tip portion 2804, the proximal element 2806 is caused to slide distally away from the handle 2802 and towards the distal element 2807, whereas when the third knob 2820 is moved away from the tip portion 2804 the proximal element 2806 is caused to slide proximally towards the handle portion 2802 and away from the distal element 2807.

In some embodiments, the proximal element 2806 expands to a diameter depending upon an application/functional use of the apparatus 2800. By way of example, for use in treatment of a pulmonary/large vessel having a diameter of up to approximately 20.0 mm, the diameter of an expanded proximal element 2806 ranges from approximately 10.0 mm to 25.0 mm; for use in treatment of a peripheral/DVT vessel having a diameter ranging from approximately 5.0 mm to 10.0 mm, the diameter of an expanded proximal element 2806 ranges from approximately 3.0 mm to 12.0 mm; for use in treatment of neuro vessels, the diameter of an expanded proximal element 2806 ranges from approximately 2.0 mm to 10.0 mm; for use in retrieval of an occlusion in the inferior vena cava (IVC) vessels, the diameter of an expanded proximal element 2806 ranges from approximately 35.0 mm to 40.0 mm.

In some embodiments, the second slider 2818 locks (and thus, cannot be moved further forward) in a position in the groove 2812 when the proximal element 2806 has expanded to a maximum diameter. Thus, sliding the second slider 2818 forward enables the user to expand the proximal element 2806 to a plurality of intermediate diameters and up to a maximum permissible diameter.

In some embodiments, the second slider 2818 is provided with a “clutch” feature so that, when opposing pressure is experienced from walls of a blood vessel during expansion of the proximal element 2806, the “clutch” provides an audio “click” sound, so that the user does not over expand the proximal element 2806. This feature is advantageous since it prevents the user from damaging the blood vessel due to over expansion of the proximal element 2806.

In some embodiments, the third slider 2820 may be configured to move synchronously along with the second slider 2818 toward the tip portion 2804 and/or in an opposing direction away from the tip portion 2804 for dislodging an occlusion and placing it in the distal element 2807.

In some embodiments, the groove 2812 has a series of interlocking features along its length such that the second slider 2818 can be selectively engaged or disengaged from a locked position in the handle 2802 at a plurality of expanded diameters for the proximal element 2806.

In some embodiments, the retrieval apparatus 2800 utilizes a lead-screw mechanism for continuous adjustment of the diameters of the proximal and distal elements 2806, 2807, so that the corresponding second slider 2818 and first slider 2814 may be advanced or retracted to an infinitely variable number of positions in the groove 2812 and may be held in a desired position by using a friction based locking mechanism, in order for the proximal and distal elements 2806, 2807 to attain a desired diameter.

In some embodiments, a non-back driving thread pattern in the lead-screw is used to provide a friction-brake when not actuated by the user, enabling continuous adjustment of the diameters of expanded proximal and distal elements 2806, 2807.

In some embodiments, the fourth tube 2816 and the third tube 2827 are telescoped together to cause the distal element 2807 to expand or contract, and the second tube 2825 and the first tube 2830 are telescoped together, to cause the proximal element 2806 to expand or contract.

In some embodiments, by moving the third slider 2820, leading to advancing or retracting of the second tube 2825 and the first tube 2830 together as one, the relative positions of the proximal element 2806 and the distal element 2807 can be adjusted in an expanded or collapsed state.

In an alternate embodiment, by moving the third slider 2820, which leads to advancing or retracting of the third tube 2827 and the fourth tube 2816 together as one, the relative positions of the proximal element 2806 and the distal element 2807 can be adjusted in an expanded or collapsed state.

In some embodiments, a distance between the proximal element 2806 and the distal element 2807 ranges between approximately 2.0 mm to 60.0 mm. Stated differently, the third knob 2820 can be actuated to cause the proximal element 2806 to move distally towards the distal element 2807 until a minimum distance between the proximal and distal elements 2806, 2807 is approximately 2.0 mm.

Similarly, the third slider can be actuated to cause the proximal element 2806 to move proximally and away from the distal element 2807 until a maximum distance between the proximal and distal elements 2806, 2807 is approximately 60.0 mm.

In some embodiments, the second slider 2818 can be positioned at several different locations/positions along the length of the groove 2812, wherein each of the locations/positions corresponds to a different degree of expansion of the proximal element 2806, and, hence, a different shape of the proximal element 2806.

In some embodiments, a minimum distance between the proximal and distal elements 2806, 2807 ranges from approximately 0.0 mm to 5.0 mm and a maximum distance between the proximal and distal elements 2806, 2807 ranges from approximately 60.0 mm to 400.0 mm.

In some embodiments, the first tube 2830 extends from the handle portion 2802 to the proximal element 2806 and is co-axial with the second tube 2825, while the third tube 2827 extends from the handle portion 2802 to the distal element 2807 and is co-axial with the fourth tube 2816, which is fixedly attached to the anchor nose 2834.

In some embodiments, the second tube 2825 and the fourth tube 2816 provide a fixed distal position of the corresponding wire mesh (proximal or distal elements 2806, 2807 respectively) against which the telescoping first tube 2830 and the third tube 2827 actuate to expand the proximal or distal elements 2806, 2807, respectively.

Preferably, the anchor nose 2834 provides a termination and fixation point for the distal element 2807 and, in some embodiments, performs a secondary function of a radiopaque marker, i.e., a reference function that provides visual guidance for an operator.

In some embodiments, diameters of the telescoping tubes 2830, 2825, 2827 and 2816 range from approximately 0.10 mm to 1.0 mm for neurovascular and peripheral applications, and approximately 1.0 mm to 3.0 mm for pulmonary and larger applications.

In some embodiments, the fourth tube 2816 may be a solid wire instead of a hollow tube.

According to the invention, a fully expanded distal element 2807 can comprise a concave shape or can be shaped like a chalice, cup, or a half-sphere, as shown in FIGS. 28E and 28F, while in a less expanded state the distal element 2807 can take different shapes.

Referring back to FIGS. 28A and 28H, in some embodiments, in order to retrieve an occlusion from a lumen of a patient, the delivery catheter 2848 is positioned near the occlusion (using the handle 2802) and the tip portion 2804 is positioned within, or all the way through, the occlusion. Once the occlusion matter is captured between the proximal element 2806 and the distal element 2807, the syringe 2837 is actuated to generate suction at an aspiration line 2824 and aspirate the occlusion matter through the aspiration catheter 2835.

Thus, in some embodiments, the proximal and distal elements 2806, 2807 expand to a particular diameter and a particular radial force, thereby allowing trapping and curettage of an occlusion, i.e., thrombus or clot material, from a vessel lumen and wall.

In some embodiments, the retrieval apparatus 2800 utilizes its adjustable radial forces and its adjustable size to actively curettage the wall of an artery or vein.

In some embodiments, the retrieval apparatus 2800 enables removal of thrombus by simultaneously capturing, compressing, dragging and curetting thrombotic material from vessel walls.

In some embodiments, the proximal element is configured to capture, and/or contain, a size of an occlusion, i.e., thrombus or clot material, in a volume range of approximately 1.0 mm to 100.0 cm.

In some embodiments, the handle portion 2802 includes a plurality of gradations such as, for example and by way of example only, three gradations of low, medium and high, five gradations ranging from low to high or eight gradations ranging from low to high. Each gradation is indicative of a corresponding predefined diameter of the proximal and distal elements in expanded states. The three slide buttons 2814, 2818, 2820 can be actuated to any one of the plurality of gradations and then détente to that position.

As indicated above, in some embodiments, the handle portion 2802 includes three buttons 2814, 2818, 2820 to manipulate the proximal and distal elements 2806, 2807, in alternate embodiments fewer than three buttons may be used.

By way of example, in some embodiments, a clinician's use of the retrieval apparatus 2800 is monitored over a predefined number of uses or operations of the apparatus 2800 while performing mechanical thrombectomy procedures. Based on the monitoring, a preferred sequence of deployment of the proximal and distal elements 2806, 2807 is determined and data indicative of the deployment sequence is stored in a memory (residing within the handle portion 2802 or remote from the handle portion 2802).

As a non-limiting illustration, the deployment sequence may include (after placing the apparatus 2800 proximate an occlusion) expanding the distal element first followed by expanding the proximal element 2806.

Consequently, a first button (when actuated) is programmed to carry out the deployment sequence and a second button is then used to reciprocate the proximal element axially. Thus, in this illustration, only two buttons are required to manipulate the proximal and distal elements 2806, 2807.

In another case scenario, the deployment sequence includes expanding the distal element 2807, expanding the proximal element and then moving the proximal element 2806 axially fore and aft for a cycle of 5 reciprocations. Consequently, a first button (when actuated) is programmed to carry out the deployment sequence.

In some embodiments, the second and third buttons can still be used manually after the deployment sequence has been completed by the programmed button.

In some embodiments, an Artificial Intelligence (AI) algorithm implements the deployment sequence, once the retrieval apparatus 2800 is placed in vivo, to automatically expand the proximal and distal elements 2806, 2807 and/or move the proximal element 2806 axially.

As also indicated above, the three buttons 2814, 2818, 2820 can be sliders, knobs, levers, dials, push buttons or a combination thereof. For example, first and second knobs can be used to expand/contract the proximal and distal elements 2806, 2807 respectively while a push button may be used to move the proximal element 2806 axially.

In another example, first, second and third levers can be actuated to generate pump actions to expand/contract the proximal and distal elements 2806, 2807 and to move the proximal element 2806 axially.

In yet another example, according to the invention, first and second dials can be actuated clockwise/counter-clockwise to expand/contract the proximal and distal elements 2806, 2807 respectively while a slider button may be used to move the proximal element 2806 axially.

Push buttons may use servos and ball screws to expand/contract the proximal and distal elements 2806, 2807 and to move the proximal element 2806 axially.

In some embodiments, the distal and/or proximal elements 2807, 2806 are self-expanding Nitinol™ wire meshes.

In some embodiments, the self-expanding distal and/or proximal elements 2807, 2806 are restrained by the handle portion 2802.

In some embodiments, the self-expanding distal and/or proximal elements 2807, 2806 are constrained by a sheath or ring that covers the tip portion 2804. The self-expanding distal and/or proximal elements 2807, 2806 expand when the constraining sheath or ring is removed.

Thus, in some embodiments, the self-expanding distal and/or proximal elements 2807, 2806 are configured for expansion based on removal of a constraining or resisting member.

In some embodiments, the retrieval apparatus 2800, within the catheter 2835, has a hypo tube and a central wire is positioned within the hypo tube. The distal element 2807 is positioned on the wire while the proximal element 2806 is positioned on the hypo tube at a fixed location.

After the retrieval apparatus 2800 is in place, the central wire is passed out of the catheter 2835 and the distal element 2807 becomes unconstrained and automatically pops open to a preset size or outer diameter (self-expanding).

With the central wire and distal element 2807 in place, the hypo tube is then moved axially. By virtue of central wire being positioned within the hypo tube, moving the hypo tube automatically moves the proximal element 2806 relative to the distal element 2807.

The hypo tube is then moved until the proximal element 2806 also pops open.

The physician then moves the hypo tube relative to the wire (which is fixed in place) to move the proximal element 2806 relative to the distal element 2807 and scrub out the clot/occlusion.

Additionally, since a procedure using the retrieval apparatus 2800, to remove an occlusion or unwanted material from a vessel lumen, is typically carried out under fluoroscopy, (i) the physician can see the internal diameter of the vessel lumen, feel the tactile feedback (generated due to the first slider 2814 having the plurality of teeth that allows the distal element 2807 to be opened or expanded incrementally) versus the outer diameter of the expanded distal element 2807 and (ii) be provided with at least one visual reference.

### Blood Reinfusion System

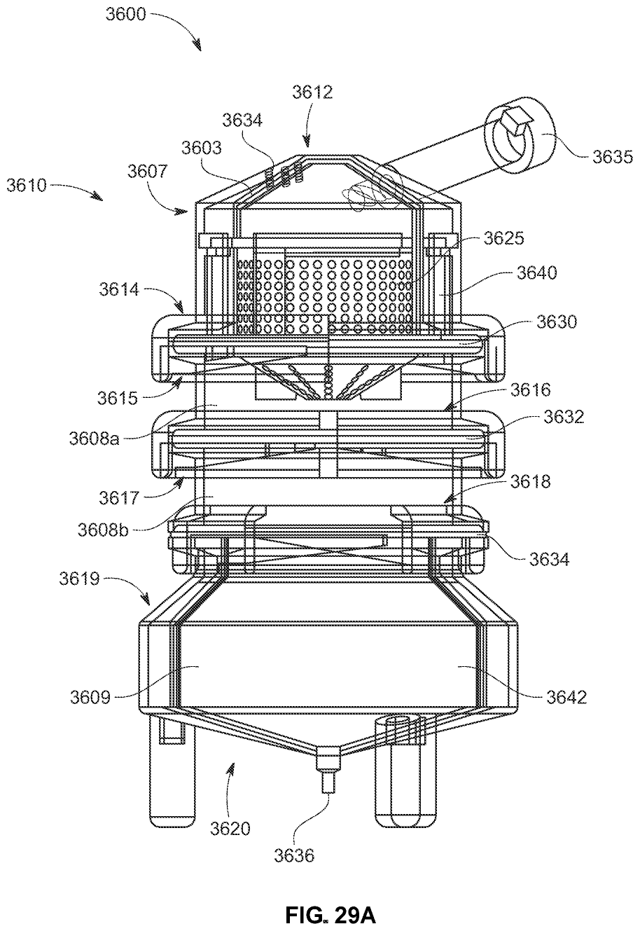

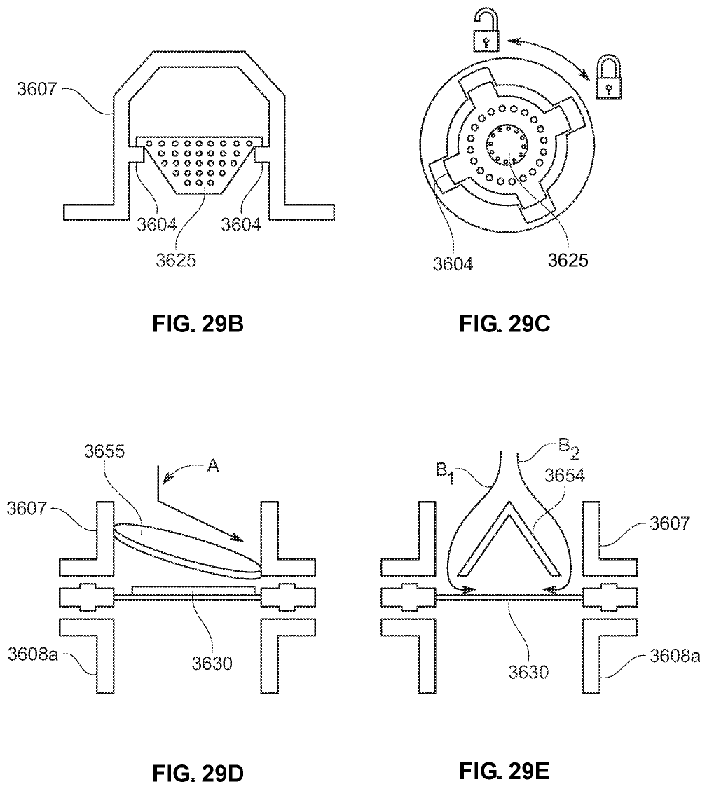

Referring now to FIG. 29A, there is depicted one embodiment of a blood reinfusion system 3600.

In a preferred embodiment, the blood reinfusion system 3600 includes at least a first suction source, such as, for example, syringe 2837 depicted in FIG. 28H and a second suction source, such as for example, syringe 3644 depicted in FIG. 28I.

As indicated above, the suction source, in this instance, syringe 2837 is coupled to and in pressure communication with the aspiration catheter 2835.

Once the retrieval apparatus 2800 dislodges the occlusion, i.e., clot or thrombus, from a vessel lumen, the syringe 2837 is actuated to apply negative pressure at a proximal end of the aspiration catheter 2835. This causes the dislodged occlusion and blood to be introduced into the syringe 2837.

In order to prevent loss of blood due to aspiration of the occlusion, i.e., clot, it is desired to reinfuse the aspirated blood back into the patient after removing the clot from the aspirated blood, with a minimum number of attachment and/or detachment steps.

In a preferred embodiment, the blood reinfusion system 3600 is utilized to remove an occlusion, i.e., clot, material from the aspirated mixture of clot material and blood and reinfuse filtered blood back into the patient.

As illustrated in FIG. 29A, in some embodiments, the blood reinfusion system 3600 comprises a substantially vertical housing 3610 having a top housing portion 3607, a first intermediate housing portion 3608a, a second intermediate housing portion 3608b and a bottom housing portion 3609.

As further illustrated in FIG. 29A, the top housing portion 3607 has a first end 3612 and a second end 3614, the first intermediate housing portion 3608a has a third end 3615 and a fourth end 3616, the second intermediate housing portion 3608b has a fifth end 3617 and a sixth end 3618 while the bottom housing portion 3609 has a seventh end 3619 and an eighth end 3620.

In some embodiments, a vertical height of the system 3600 is in a range of approximately 100.0 mm to 300.0 mm.

In some embodiments, the vertical height of the system 3600 is in the range of approximately 175.0 mm to 200.0 mm.

In some embodiments, the outer walls of the top housing portion 3607 and bottom housing portion 3609 are tapered to minimize dead space within the housing portions.

In some embodiments, the top housing portion 3607 and bottom housing portion 3609 are substantially cylindrical or cubical.

According to the invention, the first end 3612 and eighth end 3620 can be shaped in the form of bone-ends, spheroids, semi-spheroids or cuboids.

In some embodiments, the first end 3612 is dome-shaped.

In some embodiments, the first and second intermediate housing portions 3608a, 3608b are substantially cylindrical.

In some embodiments, the second end 3614, third end 3615, fourth end 3616, fifth end 3617, sixth end 3618 and seventh end 3619 have mating screws so that the top housing portion 3607, the first intermediate housing portion 3608a, the second intermediate housing portion 3608b and the bottom housing portion 3609 can be unscrewed and detached from one another or screwed together so that they are attached to one another.

In some embodiments, the top housing portion 3607 includes a first reservoir 3640 that is adapted and configured to receive a mixture of clot material and blood and the bottom housing portion 3609 includes a second reservoir 3642 that is adapted and configured to receive filtered blood.

In some embodiments, the first reservoir 3640 has a volume in a range of approximately 60.0 ml to 300.0 ml, while the second reservoir 3642 has a volume in a range of approximately 100.0 ml to 400.0 ml.

In some embodiments, the housing portions 3607, 3608a, 3608b and 3609 are made of clear or transparent material so that the contents of the first reservoir 3640 and second reservoir 3642 (as well as those of the housing portions 3608a, 3608b) are visible.

In some embodiments, the top housing portion 3607, the first intermediate housing portion 3608a, the second intermediate housing portion 3608b and the bottom housing portion 3609 are detachable from one another for ease of access and cleaning of the respective inner portions.

In some embodiments, the first end 3612 includes a first inlet port 3635 and an air vent 3634 while the eighth end 3620 includes a second outlet port 3636; the first inlet port 3635 preferably being positioned in a side wall 3603 of the top housing portion 3607 (that is, away from a central longitudinal vertical axis of the system 3600) to prevent a flow of injected clot material and blood from directly impacting the perforation of a first filter 3625.

In some embodiments, the first inlet port 3635 comprises a first luer connector to enable the first syringe 2837 to be detachably coupled with and be in fluid communication with the first inlet port 3635.

In some embodiments, the second outlet port 3636 has a flexible tube and second (female) luer connector to enable a second syringe 3644 to be detachably coupled with and be in fluid communication with the second outlet port 3636.

In accordance with some aspects, the system 3600 includes a first filter (perforated basket) 3625, a second filter or gasket 3630, a third filter or gasket 3632, and a fourth filter or gasket 3634.

In accordance with some aspects, the system 3600 further includes a fifth and final filter or gasket that is disposed in the bottom housing portion 3609, more preferably, in the second reservoir 3642 thereof.

In some embodiments, the third filter or gasket 3632 has a pore size of approximately 250.0 μm and the fourth filter or gasket 3634 has a pore size of approximately 40.0 μm.

As depicted in FIGS. 29B and 29C, in some embodiments, the inner walls of the top housing portion 3607 include channels 3604 that allow for the first filter 3625, when inserted into the top housing portion 3607 from the bottom of the top housing portion 3607 (when the housing portion 3607 has been detached from the intermediate housing portion 3608), to be twisted in a first direction and be locked in place, and twisted in a second direction (opposite to the first direction) to unlock.

Referring back to FIG. 29A, in some embodiments, each of the second filter or gasket 3630, third filter or gasket 3632, and fourth filter or gasket 3634 are enclosed in respective first, second, third and fourth O-ring seals.

According to the invention, the first filter 3625 can include an O-ring.

In a preferred embodiment, the first filter 3625 does not include an O-ring.

In some embodiments, the first filter 3625 has a pore size in the range of approximately 1.0 mm to 5.0 mm, more preferably, a pore size of approximately 2.0 mm.

In some embodiments, the second filter 3630 has pore size in the range of approximately 200.0 micron to 1.0 mm, more preferably, a pore size of approximately 1.0 mm.

In some embodiments, the third filter 3632 has pore size in the range of approximately 40.0 micron to 1.0 mm, more preferably, a pore size of approximately 200.0 micron.

In some embodiments, the fourth filter 3634 has pore size in a range of approximately micron to 40.0 micron, more preferably, a pore size of approximately 30.0 micron.

In some embodiments, the fifth and final filter has a pore size of approximately micron or less.

In some embodiments, the final filter pore size is the same as a standard inline blood transfusion filter.

One objective of configuring multiple filters is to prevent the filters from clogging. In some embodiments, the second filter or gasket 3630 is removed, without affecting the results.

In some embodiments, the first filter 3625 is coarser, compared to the remaining filters/gaskets, in order to capture or trap majority of large chunks of thrombus with progressively smaller thrombi being captured through each successive filter.

In some embodiments, a flow redirector element 3655 is positioned above and proximate each of the first filter 3625, the second filter 3630, third filter 3632, and fourth filter 3634 to bias and control a direction of flow of clot material and blood (denoted by Arrow A) towards a side or portion of each of the filters, see, e.g., FIG. 29D, which depict a flow redirector element 3655 positioned above and proximate the second filter 3630. By doing so, thrombi accumulation is focused to a portion of each of the filters while the remaining portion of each of the filters remains open and unobstructed.

According to the invention, a plane of the flow redirector element 3655 is inclined by a predefined angle to a horizontal plane.

In some embodiments, the predefined angle of the flow redirector element 3655 ranges from approximately 0.0 degrees to 60.0 degrees from the horizontal.

In some embodiments, the flow redirector element 3655 comprises a disc-shaped element having a diameter smaller than the diameter of each of the associated second, third and fourth filters 3630, 3632, 3634.

Referring now to FIG. 29E, there is depicted a further embodiment of a flow redirector element 3654 positioned above and proximate the second filter 3630. The flow redirector element 3654 is similarly configured to bias and control direction of flow of clot material and blood towards a side or portion of each of the filters, as shown in FIG. 29E, which depicts the flow of clot material and blood towards a side or portion of the second filter 3630 (denoted by Arrows B₁ and B₂).

In some embodiments, the first filter 3625 is positioned within the top housing portion 3607, the second filter 3630 is positioned between the second end 3614 and the third end 3615 such that it lies between the top housing portion 3607 and the first intermediate housing portion 3608a, the third filter 3632 is positioned between the fourth end 3616 and fifth end 3617 such that it lies between the first intermediate housing portion 3608a and the second intermediate housing portion 3608b, and the fourth filter 3634 is positioned between the sixth end 3618 and seventh end 3619 so that the fourth filter 3634 is positioned between the second intermediate housing portion 3608b and the bottom housing portion 3609.

It should be appreciated that the respective first, second, third and fourth O-rings of the first filter 3625, second filter 3630, third filter 3632, and fourth filter 3634 provide fluidic seals.

For assembly, the fourth filter 3634 with the associated fourth O-ring is placed on the seventh end 3619 and then the second intermediate housing portion 3608b is attached (via a screwing mechanism) to the bottom housing portion 3609.

Thereafter, the third filter 3632 with the associated third O-ring is placed on the fifth end 3617 and then the first intermediate housing portion 3608a is attached (via a screwing mechanism) to the second intermediate housing portion.

Thereafter, the second filter 3630 with the associated second O-ring is placed on the third end 3615 while the first filter 3625 is positioned within the top housing portion 3607 and then the top housing portion 3607 is attached (via a screwing mechanism) to the first intermediate housing portion 3608a.

In some embodiments of the invention, the method for retrieving a mixture of blood and clot material from a vessel of a patient and isolating the blood from the mixture for reinfusion into the patient initially comprises steps of providing one of the aforementioned retrieval apparatus of the invention, such as the retrieval apparatus depicted in FIG. 28A, an aspiration catheter, in this instance, aspiration catheter 2835 depicted in FIG. 28A, and the blood reinfusion system 3600 described above.

After the retrieval system, aspiration catheter 2835 and blood reinfusion system 3600 are provided, the clot material is dislodged from the vessel with the retrieval system.

After the clot material is dislodged from the vessel, the aspiration catheter 2835 is positioned at a treatment site proximate the dislodged clot material in the vessel.

Thereafter, a first syringe, such as syringe 2837 depicted in FIG. 28H, is coupled to the aspiration catheter 2835 and the first syringe 2837 is employed to extract a mixture of the dislodged clot material and blood from the vessel.

After the dislodged clot material and blood is extracted from the vessel with the first syringe 2837, the first syringe 2837 (containing the mixture of dislodged clot material and blood) is decoupled from the aspiration catheter 2835 and coupled (via the first luer connector) to the first inlet port 3635 of the blood reinfusion system 3600.

After the first syringe 2837 is coupled to the first inlet port 3635 of the blood reinfusion system, the mixture of clot material and blood contained in the first syringe 2837 is introduced into the first reservoir 3640 of the blood reinfusion system 3600.

According to the invention, after the mixture of dislodged clot material and blood is introduced into the blood reinfusion system 3600, the mixture of clot material and blood passes from the first reservoir 3640, preferably due to gravity, (i) into and through the first filter 3625 of the blood reinfusion system 3600, wherein first filtered blood is obtained, (ii) into and through the second filter 3630 of the blood reinfusion system 3600, wherein second filtered blood is obtained, (iii) into and through the third filter 3632 of the blood reinfusion system 3600, wherein third filtered blood is obtained, and (iv) into and through the fourth filter 3634 of the blood reinfusion system 3600, wherein fourth filtered blood is obtained.

After the third filtered blood passes through the fourth filter 3634 and the fourth filtered blood is obtained, the fourth filtered blood passes into the second reservoir 3642 of the blood reinfusion system 3600.

For reinfusion of blood into the patient, the second syringe 3644 depicted in FIG. 28I is coupled to the second outlet port 3636 (via the second luer connector) and the fourth filtered blood is aspirated from the second reservoir 3642.

After the fourth filtered blood is aspirated from the second reservoir 3642 of the blood reinfusion system 3600, a second syringe, such as syringe 3644 depicted in FIG. 28I, is decoupled from the second outlet port 3636 and connected to the patient's infusion line to infuse the filtered blood, i.e., fourth filtered blood, back to the patient.

According to the invention, in the event that a practitioner, e.g., surgeon, does not effectively dislodge and/or aspirate all of the clot material from the vessel via the method steps above, the above procedure from the dislodgement of the clot material through the introduction of the mixture of clot material and blood into the first reservoir 3640 of the blood reinfusion system 3600 can be repeated.

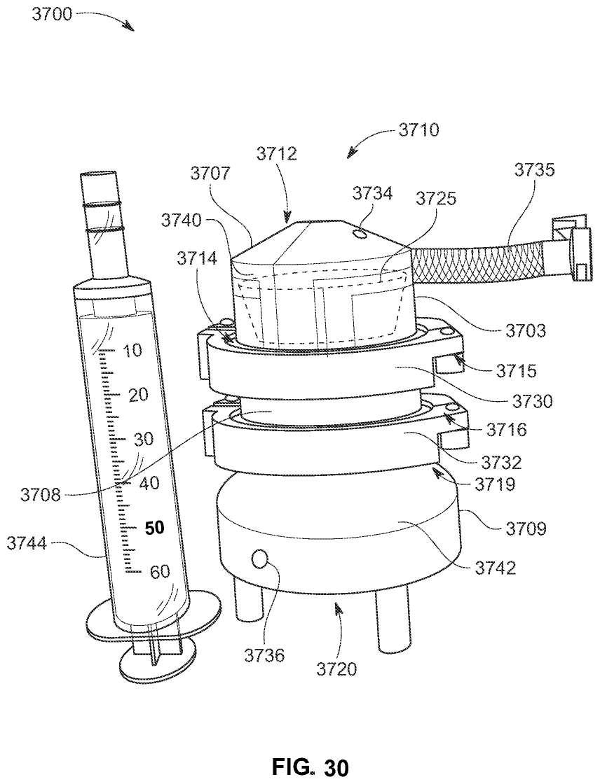

Referring now to FIG. 30, there is depicted yet another blood reinfusion system 3700 for use with the retrieval apparatus 2800.

As depicted in FIG. 30, in some embodiments, the system 3700 comprises a substantially vertical housing 3710 having a top housing portion 3707, an intermediate housing portion 3708 and a bottom housing portion 3709.

As further depicted in FIG. 30, the top housing portion 3707 has a first end 3712 and a second end 3714, the intermediate housing portion 3708 has a third end 3715 and a fourth end 3716, while the bottom housing portion 3709 has a fifth end 3719 and sixth end 3720.

In some embodiments, the outer walls of the top housing portion 3707 and bottom housing portion 3709 are similarly tapered to minimize dead space within the said housing portions.

In some embodiments, the top and bottom housing portions 3707, 3709 are substantially cylindrical or cubical.

According to the invention, the first end 3612 and sixth end 3720 can be shaped in the form of bone-ends, spheroids, semi-spheroids or cuboids.

In some embodiments, the first end 3712 is dome-shaped.

In some embodiments, the intermediate housing portion 3708 is substantially cylindrical.

In some embodiments, the second end 3714 third end 3715, fourth end 3716, and fifth end 3719 have mating screws so that the top housing portion 3707, the intermediate housing portion 3708 and the bottom housing portion 3709 can be unscrewed and detached from one another or screwed to be attached back to one another.

In some embodiments, the top housing portion 3707 includes a first reservoir 3740 that is adapted to receive a mixture of clot material and blood and the bottom housing portion 3709 includes a second reservoir 3742 that is adapted and configured to store filtered blood.

In some embodiments, the first reservoir 3740 has a volume in the range of approximately 60.0 ml to 300.0 ml while the second reservoir 3742 has a volume in a range of approximately 100.0 ml to 400.0 ml.

In some embodiments, the housing portions 3707, 3708 and 3709 are made of clear or transparent material so that the contents of the first reservoir 3740 and second reservoir 3742 (as well as those of the housing portion 3708) are visible.

In some embodiments, the top housing portion 3707, the intermediate housing portion 3708 and the bottom housing portion 3709 are detachable from one another for ease of access and cleaning of the respective inner portions.

In some embodiments, the first end 3712 includes a first inlet port 3735 and an air vent 3734 while the sixth end 3720 includes a second outlet port 3736.

In some embodiments, the first inlet port 3735 is positioned in the side wall 3703 of the top housing portion 3707 (that is, away from a central longitudinal vertical axis of the system 3700) to prevent a flow of injected clot material and blood from directly impacting the perforation of a first filter 3725.

In some embodiments, the first inlet port 3735 has a first luer connector to enable a first syringe, such as syringe 2837, to be detachably coupled with and be in fluid communication with the first inlet port 3735.

In some embodiments, the second outlet port 3736 has a second luer connector to enable a second syringe, such as syringe 3744, to be detachably coupled with and be in fluid communication with the second outlet port 3736.

In accordance with some aspects, the system 3700 includes first filter 3725, second filter 3730 and third filter 3732.

In some embodiments, the inner walls of the top housing portion 3707 include threads therein that allow for the first filter 3725, when inserted into the top housing portion 3707 from the bottom of the top housing portion 3707 (when the housing portion 3707 has been detached from the intermediate housing portion 3708), to be twisted in a first direction and be locked in place, and twisted in a second direction (opposite to the first direction) to unlock.

In some embodiments, each of the second and third filters 3730, 3732 are enclosed in respective second and third O-ring seals.

In some embodiments, the first filter 3725 has a pore size in the range of approximately 1.0 mm to 5.0 mm, the second filter 3730 has a pore size in the range of approximately 200.0 micron to 1.0 mm, and the third filter 3732 has a pore size in the range of approximately micron to 1.0 mm.

In some embodiments, the first filter 3725 is coarser, compared to the remaining filters, in order to capture or trap majority of large chunks of thrombus with progressively smaller thrombus being captured through each successive filter.

In some embodiments, the diameter of the first filter 3725 is approximately 76.0 mm for improved filtration and prevention of the successive filters from becoming clogged.

In some embodiments, the first filter 3725 comprises a 2.0 mm pore filter in the shape of a basket, the second filter 3730 comprises a 1.0 mm pore filter, and the third filter 3732 comprises a 200.0 micron pore filter.

In yet another embodiment, three filters are used, wherein the first filter comprises a 2.0 mm pore filter in the shape of a basket, the second filter comprises a 200.0 micron pore round filter, and the third filter 3634 comprises a 40.0 micron pore round filter.

In some embodiments, a flow redirector element is positioned above and proximate the first filter 3725 to bias and control the direction of flow of clot material and blood towards a side or portion of the filter. By doing so, thrombi accumulation is directed to a portion of each of the filters while the remaining portion of each the filters remains open and unobstructed.

In some embodiments, a plane of a flow redirector element, such as flow redirector element 3655, is inclined by a predefined angle to a horizontal plane.

In some embodiments, the predefined angle similarly ranges from approximately 0.0 degrees to 60.0 degrees from the horizontal.

In some embodiments, the flow redirector element is a disc shaped element having a diameter smaller than that of each of the associated second and third filters 3730, 3732.

In embodiments, the first filter 3725 is positioned within the top housing portion 3707, the second filter 3730 is positioned between the second end 3714 and third end 3715 such that it lies between the top housing portion 3707 and the intermediate housing portion 3708, and the third filter 3732 is positioned between the fourth end 3716 and fifth end 3719 such that it lies between the intermediate housing portion 3708 and the bottom housing portion 3709.

For assembly, the third filter 3732 with the associated third O-ring is placed on the fifth end 3719 and the intermediate housing portion 3708 is attached (i.e., by a screwing action) to the bottom housing portion 3709. Thereafter, the second filter 3730 with the associated second O-ring is placed on the third end 3715 while the first filter 3725 is positioned within the top housing portion 3707 and the top housing portion 3707 is attached (i.e., by a screwing action) to the intermediate housing portion 3708.

For filtration, the first syringe, in this instance, syringe 2837, containing a mixture of clot material and blood is detached from the aspiration catheter 2835 and coupled (via the first luer connector) to the first inlet port 3735 to inject the mixture of clot material and blood into the first reservoir 3740 of the top housing portion 3707.

The mixture of clot material and blood from the first reservoir 3740 then passes, preferably due to gravity, through the first filter 3725 in the top housing portion 3707, the second filter 3730 into the intermediate housing portion 3708 and the third filter 3732 into the second reservoir 3742.

Consequently, clot material and related debris is filtered out while filtered blood collects in the second reservoir 3742 due to gravity.

For reinfusion of blood to the patient, the second syringe, in this instance, syringe 3744, is coupled to the second outlet port 3736 (via the second luer connector) to aspirate filtered blood from the second reservoir 3742.

Once filled, the second syringe 3744 is detached from the second outlet port 3736 and connected to a patient's infusion line to infuse filtered blood back to the patient.

Although only a few embodiments of the present invention have been described herein, it should be understood that the present invention can be embodied in many other specific forms without departing from the spirit or scope of the invention. Therefore, the present example and embodiments are to be considered as illustrative and not restrictive, and the invention can be modified within the scope of the appended claims.

1. A method for retrieving a mixture of clot material and blood from a vessel of a patient and isolating the blood from the mixture for reinfusion into the patient, comprising the steps of:
providing an aspiration catheter configured and adapted to be positioned in a first vessel of said patient;
providing first and second suction sources, said first suction source adapted to couple to said aspiration catheter and aspirate a first mixture of first clot material and first blood from said first vessel of said subject when said first suction source is coupled to said aspiration catheter;
providing a blood reinfusion system adapted to isolate said first blood from said first mixture of said first clot material and said first blood,
said blood reinfusion system comprising filter means for filtering said first mixture of said first clot material and said first blood from said first vessel of said subject, said filter means comprising a first filter, a second filter, a third filter and a fourth filter,
said blood reinfusion system further comprising a top housing portion, a first intermediate housing portion, a second intermediate housing portion and a bottom housing portion,
said top housing portion of said blood reinfusion system comprising a first reservoir and an inlet port, said first reservoir adapted to receive said first mixture of said first clot material and said first blood, said inlet port adapted to couple said top housing portion of said blood reinfusion system to said first suction source,
said first filter positioned in said top housing portion of said blood reinfusion system,
said second filter positioned between said top housing portion of said blood reinfusion system and said first intermediate housing portion of said blood reinfusion system,
said third filter positioned between said first intermediate housing portion of said blood reinfusion system and said second intermediate housing portion of said blood reinfusion system,
said fourth filter positioned between said second intermediate housing portion of said blood reinfusion system and said bottom portion of said blood reinfusion system,
said bottom housing portion of said blood reinfusion system comprising a second reservoir and an outlet port, said second reservoir adapted to receive filtered blood from said filter means of said blood reinfusion system, said outlet port adapted to couple said configured to connect bottom housing portion to said second suction source;
coupling said first suction source to said aspiration catheter;
positioning said aspiration catheter at a treatment site proximate said first clot material within said first vessel of said patient;
aspirating said first mixture of said first clot material and said first blood from said first vessel with said first suction source, wherein said first mixture of said first clot material and said first blood is introduced into and through said aspiration catheter and into said first suction source;
decoupling said first suction source from said aspiration catheter;
coupling said first suction source to said inlet port of said blood reinfusion system;
injecting said first mixture of said first clot material and said first blood in said first suction source into and through said inlet port and into said first reservoir of said blood reinfusion system, wherein said first mixture of said first clot material and said first blood passes through said first filter of said blood reinfusion system and a first filtered blood is obtained, said first filtered blood passes through said second filter of said blood reinfusion system and a second filtered blood is obtained, said second filtered blood passes through said third filter and a third filtered blood is obtained, said third filtered blood passes through said fourth filter and a fourth filtered blood is obtained, and said fourth filtered blood passes into said second reservoir of said blood reinfusion system;
coupling said second suction source to said outlet port of said blood reinfusion system; and
aspirating said fourth filtered blood from said second reservoir with said second suction source, wherein said fourth filtered blood is introduced into said second suction source for reinfusion into said patient.

2. The method of claim 1, wherein said first suction source comprises a first syringe.

3. The method of claim 1, wherein said second suction source comprises a second syringe.

4. The method of claim 1, wherein said second filter comprises a pore size in a range of 200.0 micron to 1.0 mm.

5. The method of claim 1, wherein said third filter comprises a pore size in a range of 40.0 micron to 1.0 mm.

6. The method of claim 1, wherein said fourth filter comprises a pore size in a range of 10.0 micron to 40.0 micron.

7. The method of claim 1, wherein said blood reinfusion system further comprises a flow redirector element positioned above and proximate at least one of said first filter, said second filter, said third filter, and said fourth filter.

8. The method of claim 1, wherein said method further comprises the step of providing a retrieval system adapted to dislodge said first clot material from said first vessel of said patient.

9. The method of claim 8, wherein said retrieval system comprises an elongated member, said elongated member comprising a longitudinal axis, a distal end region, a first segment and a second segment, said first segment of said elongated member adapted to move axially independent of said second segment of said elongated member,
said elongated member further comprising a distal element and a proximal element,
said distal element comprising a first proximal end point and a first distal end point, said first proximal end point engaged to a first portion of said second segment of said elongated member, said first distal end point engaged to a second portion of said second segment of said elongated member, said first and second portions of said second segment of said elongated member disposed proximate said distal end region of said elongated member,
said distal element adapted to expand from a first pre-expansion configuration to a first expanded configuration, and contract from said first expanded position to a first contracted configuration,
said proximal element comprising a second proximal end point and a second distal end point, said second proximal end point of said proximal element engaged to a first portion of said first segment of said elongated member and said second distal end point of said proximal element engaged to a second portion of said first segment of said elongated member,
said proximal element adapted to move axially in a first direction coincident with said longitudinal axis of said elongated member when said first segment of said elongated member is said moved axially,
said proximal element adapted to expand from a second pre-expansion configuration to a second expanded configuration, and contract from said second expanded position to a second contracted configuration,
said retrieval sub-system further comprising a handle in physical communication with said elongated member, said handle comprising a first physically manipulable interface, a second physically manipulable interface and a third physically manipulable interface,
said first physically manipulable interface adapted to mechanically induce said expansion of said proximal element from said second pre-expansion configuration to said second expanded configuration and said contraction of said proximal element from said second expanded position to said second contracted configuration, when said first physically manipulable interface is moved,
said second manipulable interface adapted to mechanically induce said expansion of said distal element from said first pre-expansion configuration to said first expanded configuration and said contraction of said distal element from said first expanded position to said first contracted configuration, when said second physically manipulable interface is moved,
said third physically manipulable interface adapted to induce said axial movement of said first segment of said elongated member, when said third physically manipulable interface is moved.

10. The method of claim 1, wherein said top housing portion of said blood reinfusion system is adapted to allow said first filter to be twisted in a first direction to be locked in a first position to said top housing portion, and twisted in a second direction, opposite to said first direction, to be unlocked from said first position.

11. The method of claim 1, wherein said top housing portion is detachably attached to said first intermediate housing and said second intermediate housing portion.

12. The method of claim 1, wherein said second intermediate housing portion is detachably attached to said bottom housing portion.

13. A blood reinfusion system, comprising:
first suction means for aspirating blood from a patient, said blood comprising foreign particulates;
filter means for isolating said foreign particulates from said aspirated blood, whereby filtered aspirated blood is obtained,
said filter means comprising first connection means for coupling said filter means to said first suction means, whereby said aspirated blood is delivered to said first filter means when said filter means is coupled to said first suction means;
reservoir means in communication with said filter means for collecting said filtered aspirated blood from said filter means; and
second suction means for aspirating said filtered aspirated blood from said reservoir means for reinfusion into said patient, said reservoir means comprising second connection means for coupling said second suction means to said reservoir means.

14. The blood reinfusion system of claim 13, wherein said system further comprises housing means for enclosing said filter means and said reservoir means.

15. The blood reinfusion system of claim 13, wherein said filter means comprises a plurality of filters adapted to successively isolate said foreign particulates from said aspirated blood.

16. The blood reinfusion system of claim 15, wherein said first plurality of blood filters are interconnected.

17. The blood reinfusion system of claim 15, wherein said foreign particulates are said successively isolated from said aspirated blood when said aspirated blood is drawn through said plurality of filters by a force of gravity.

18. The blood reinfusion system of claim 13, wherein said system further comprises first conduit means for conducting said aspirated blood from said suction means to said first connection means of said filter means.

19. The blood reinfusion system of claim 13, wherein said foreign particulates comprise material selected from the group consisting of emboli and tissue debris.
